(19) United States
(12) Patent Application Publication
(10) Pub. No.: US 2013/0072837 A1
(43) Pub. Date: Mar. 21, 2013

(54) SLEEVES FOR ACCOMODATING A CIRCULATION ENHANCEMENT DEVICE

(71) Applicants: Benny Rousso, Rishon Le ZION (IL); Eliahu Eliachar, Haifa (IL); Yuval Avni, Yafo (IL); Michal Taichman, Kfar Saba (IL); Nir Lilach, Kfar Yehoshua (IL)

(72) Inventors: Benny Rousso, Rishon Le ZION (IL); Eliahu Eliachar, Haifa (IL); Yuval Avni, Yafo (IL); Michal Taichman, Kfar Saba (IL); Nir Lilach, Kfar Yehoshua (IL)

(73) Assignee: Flomedic Limited, Hamilton (BM)

(21) Appl. No.: 13/674,122

(22) Filed: Nov. 12, 2012

Related U.S. Application Data
(62) Division of application No. 11/569,808, filed on Nov. 30, 2006.

(30) Foreign Application Priority Data
Jun. 6, 2004 (IL) 162373
Mar. 29, 2005 (IL) 167734

(51) Int. Cl. A61H 1/00

(52) U.S. Cl. CPC A61H 1/008
USPC 601/152

(57) ABSTRACT

A sleeve for use with a circulation enhancement device, the sleeve substantially encircles a user's leg at the region of the user's calf muscle and includes two end-straps of non-stretchable material configured to connect to opposite lateral sides of the circulation enhancement device to enable intermittent tightening and relaxing of the sleeve around the user's leg by pulling and releasing both end straps, the two end straps extend in opposite directions along a common longitudinal axis. At least one of the two end straps is connectable to the circulation enhancement device by passing through a loop in one of the opposite lateral sides of the circulation enhancement device; and at least one end strap includes attachment means configured to be attached at various positions along the sleeve, after passing through the loop, to enable adjusting the sleeve to fit the user's leg.

### RELATED APPLICATION

This application is a divisional filing of application Ser. No. 11/569,808 having a US filing date of Nov. 30, 2006, the disclosure of which is incorporated herein by reference.

### FIELD OF THE INVENTION

The present invention relates generally to methods and apparatus for enhancing blood and lymph flow in a user.

### BACKGROUND OF THE INVENTION

The development of a “blood clot” or stasis related disorders in a limb, specifically in the lower limbs, is a major health hazard. It may lead to local symptoms and signs such as redness, pain and swelling of the affected limb. It may also be a life hazard by sending small parts of a blood clot towards the lungs corking the circulation through the lungs (called Pulmonary Embolism), leading to reduced ability of the lungs and sometimes of the heart to function. This is accompanied by pain, shortness of breath, increased heart rate and other clinical signs and symptoms.

The development of stasis related disorders is believed to be related pathologically to Virchow's triad. More specifically, a stasis related disorders has increased incidence if three conditions are met in the vasculature; Stasis (reduced blood flow), Hypercouagulability (increased tendency of clotting in a blood vessel during normal conditions) and Endothelial damage (damage to the internal layer of the blood vessel promotes clot formation).

In the ambulatory person the muscles of the leg compress the deep venous system of the leg pushing the blood towards the heart. This phenomena is called the “muscle pump”. The muscles of the calf are traditionally implicated in the mechanism of the “muscle pump”.

During a period of immobilization, stasis is believed to be the major risk factor for the formation of stasis related disorders. Immobilization includes any period of lack of physical activity whether in the supine or sitting position e.g. bed or chair ridden persons, during long automobile trips, long flights, long working hours in the sitting position etc.

Recently the medical community named the formation of stasis related disorders during long journeys, the “travelers thrombosis”. It is believed that around 5% of manifested stasis related disorders originate during traveling. This is believed to occur due to the prolonged immobilization, especially while in the sitting position. This position further compromises blood flow due to kinking of veins in the limb during the sitting position. It was further shown that enhancing the venous blood flow (via a compressing device) during flight, reduced discomfort, limb swelling, fatigue and aching when used on flight attendants.

Limb swelling and discomfort may be present also in states of lymph stasis such as after a mastectomy and in other conditions in which lymphatic return to the heart is impaired.

Increasing the flow of blood in the limb during periods of immobility is already a proven method to prevent the formation of stasis related disorders in the limb. It secondarily prevents the formation of pulmonary embolism (PE) that commonly originates from a stasis related disorders. Increasing the venous return from the lower limb can also prevent formation of edema, pain and discomfort in the limb during periods of immobilization.

Prevention of stasis related disorders is commonly achieved via large and cumbersome devices. Most of these devices can be used only by trained medical staff. Such devices operate by either of two methods: Pneumatic/Hydraulic intermittent compressions or by direct intermittent electrical stimulation of the “muscle pump”. The Pneumatic/hydraulic devices use a sleeve or cuff with a bladder that is inflated and deflated by air or fluid compressor thus causing stimulation of the physiological “muscle pump”. The pneumatic/hydraulic devices usually require a sophisticated set of tubes and valves, a compressor, a source of fluid and a sophisticated computer control. Moreover such devices emit substantial noise while operating. The electrical stimulators work by delivering electrical impulses to the calf muscles. These devices require a sophisticated electronic apparatus and may be painful or irritating to patient.

Most existing devices aimed at preventing stasis related disorders are designed for use in the medical setting, by trained personal. Such devices are generally non-portable. Most of the devices known in the art operate using a strap that is flexible and circle the circumference of the limb treated.

Accordingly it is the object of the present invention to provide a device for the enhancement of blood and lymph flow in a limb and the prevention of stasis related disorders development during periods of immobility which simulate intermittent muscle compression of a limb and is portable, self-contained, does not rely on, but is compatible with, an external power source, and is easily carried, small, and lightweight.

It is a further object of the present invention to provide such a device which is simple to operate by a lay person without any special training in the field of medicine, is easily attached over the persons limb or attached to a sleeve or a bandage placed on the limb. Fastening a device according to the present invention is simple and requires no skill from the person placing the device on his or her limb.

A further object of the present invention is to provide such a device for the prevention of stasis related disorders that is easy to manufacture and is low cost.

### SUMMARY OF THE INVENTION

An aspect of an embodiment of the invention, relates to a sleeve for deploying and activating a circulation enhancement device, comprising an independent inner layer and an independent outer layer with a connection between them. The inner layer is adapted to enhance the grip of the user's limb by the circulation enhancement device. The outer layer is adapted to transfer a force from the circulation enhancement device to the user's limb. The connection between the inner layer and outer layer preserves a relative positioning between the two.

In some embodiments of the invention, the inner layer is manufactured from an elastic material in order to enhance its grasp of the user's limb. Alternatively or additionally, the inner layer comprises an elastic lining on its edge to enhance its grasp of the user's limb.

In some embodiments of the invention, the outer layer is manufactured from a non-elastic material in order to improve the outer layers ability to be used to squeeze and release the muscles of the user.

In some embodiments of the invention, the inner layer and outer layer are connected at a fixed position along a thin line across their width. Optionally, the inner layer and outer layer may be connected at more than one point along their length. In some embodiments of the invention, the connection is created by sewing them together along a thin line. Alternatively or additionally, the connection can be created using glue, snaps, buttons, welding or other means.

In an exemplary embodiment of the invention, the inner layer is deployed to completely encircle a user's limb. Optionally, the inner layer comprises attachment means at the ends of the inner layer to seal it around the user's limb. Optionally, the inner layer is deployed to be sealed toward the front of the user's limb.

In an exemplary embodiment of the invention, the outer layer partially, encircles the inner layer. In some embodiments of the invention, the outer layer is attached to the circumference of the external side of the inner layer with hook and loop fastener material. In some embodiments of the invention, the outer layer is tightened around the circumference of the inner layer by pulling it forward toward the sealing position of the inner layer. Alternatively, the outer layer is tightened around the circumference of the inner layer by pulling it backward away from the sealing position of the inner layer.

In some embodiments of the invention, the outer layer's position can be adjusted while a circulation enhancement device is deployed. Alternatively, the circulation device is removed in order to adjust the position of the outer layer.

An aspect of an embodiment of the invention, relates to a sleeve for deploying and activating a circulation enhancement device, comprising an interface part to interface a user's limb with two straps connected to opposite sides of the interface part. Each strap comprises attachment means and attachment reception means so that it can be folded over to grasp a buckle of the circulation enhancement device. The interface is positioned opposite the circulation enhancement device. The circulation enhancement device with the straps and the interface encircle the user's limb.

In some embodiments of the invention, the sleeve comprises an elastic loop attached to the ends of said straps, in order to assist in pulling the sleeve around the user's limb.

In some embodiments of the invention, the interface has a piece cut out from its center to enhance its contact with the user's limb. In some embodiments of the invention, the interface is flat. Alternatively, the interface is shaped like a piece of a sphere.

There is thus provided in accordance with an exemplary embodiment of the invention, a sleeve for use with a circulation enhancement device, including, a first layer that is adapted to substantially encircle a user's limb, a second layer that is adapted to interface the circulation enhancement device, a connection between the first layer and the second layer at a preset position; and wherein the second layer is adapted to be attached to various positions along the circumference of the first layer and at least partially encircle the first layer.

In an exemplary embodiment of the invention, the first layer comprises an elastic material. Optionally, the first layer comprises edges with an elastic lining along the edges. In an exemplary embodiment of the invention, the second layer comprises a non-elastic material. Optionally, the first layer and the second layer are connected by sewing. In an exemplary embodiment of the invention, the first layer and the second layer are connected by snaps. Optionally, the first layer and the second layer are connected by glue. In an exemplary embodiment of the invention, the first layer and the second layer are connected by welding. Optionally, the second layer is attached to various positions along the first layer using hook and loop fasteners. In an exemplary embodiment of the invention, the first layer comprises attachment means at its ends to seal it around a user's limb. Optionally, the attachment means are hook and loop fasteners.

In an exemplary embodiment of the invention, the attachment of the second layer is tightened by pulling it forward toward the attachment means that seal the first layer. Optionally, the attachment of said second layer is tightened by pulling it backward away from the attachment means that seal the first layer. In an exemplary embodiment of the invention, the second layer is adapted to tighten the attachment while the circulation enhancement device is deployed on the second layer. Optionally, the second layer comprises a buckle. In an exemplary embodiment of the invention, the sleeve includes attachment means to interface the circulation enhancement device.

In an exemplary embodiment of the invention, the sleeve includes a support strap which is adapted to enclose a user's limb higher up on the limb than the first layer; and an attachment which attaches between the support strap and the first layer. Optionally, the support strap is adapted to enclose the user's limb on one side of a user's joint and the first layer at least partially encircles the other side of a user's joint. In an exemplary embodiment of the invention, the support strap is adapted to enclose a user's limb at a position higher than a user's knee, and attach to the first layer which at least partially encloses lower than the user's knee. Optionally, the sleeve includes a support strap which is adapted to enclose a user's limb higher up on the limb than the first layer; and the strap is an integral part of the first layer. In an exemplary embodiment of the invention, the support strap is adapted to enclose the user's limb higher up on the limb than the user's joint and the first layer extends lower down on the limb than the user's joint. Optionally, the support strap is adapted to enclose a user's limb higher up on the limb than a user's knee, and the first layer extends lower down on the limb than the user's knee. In an exemplary embodiment of the invention, the sleeve includes joint protectors for shielding a user's joint, wherein said joint protectors are deployed under the support strap.

Additionally, there is provided according to an exemplary embodiment of the invention, a sleeve for use with a circulation enhancement device, including an interface to interface a user's limb, two straps connected to opposite sides of the interface, attachment means and attachment reception means on the same side of each strap, wherein in use the straps are inserted through a buckle on each side of the circulation enhancement device and folded such that the attachment means attach to the attachment reception means grasping the buckle; and wherein in use the straps and interface encircle the user's limb with the interface on a side of the user's limb opposite the circulation enhancement device.

In an exemplary embodiment of the invention, the sleeve includes an elastic extension attached to the ends of the straps. Optionally, the interface has a piece cut out from its center. In an exemplary embodiment of the invention, the interface is flat. Alternatively, the interface is shaped like a piece of a sphere.

### BRIEF DESCRIPTION OF THE DRAWINGS

The present invention will be understood and appreciated more fully from the following detailed description taken in conjunction with the drawings in which:

FIG. 1 is a pictorial illustration of the device of the present invention strapped to the calf of a sitting person;

FIG. 2A is a side external view of a preferred anterior box embodiment of the present device, in which squeezing the limb muscles is performed by intermittent shortening the circumference of a loop created by an assembly body and strap;

FIG. 2B is a side view illustration of a posterior box embodiment in which the assembly box is the active intermittent compressing part placed against the calf muscles;

FIG. 3A is a cross section of a device in accordance with the is embodiment of FIG. 2A, showing a first internal mechanism of the assembly box;

FIG. 3B is a top view of the device of FIG. 3A;

FIG. 3C depicts a modified mechanism of the embodiment of FIGS. 3A and 3B;

FIG. 4A is pictorial representation of an alternative mechanism for the embodiment of FIG. 2A using an electromagnetic motor, a centrally hinged rotating rectangular plate and a longitudinal bar connecting both sides of the strap;

FIGS. 4B and 4C are side and top view respectively of the embodiment presented in FIG. 4A;

FIGS. 5A and 5B depict yet another mechanism for the embodiment of FIG. 2A using an enhanced power transmission by means of an “L” shaped lever bar;

FIG. 6 is a side view of yet another embodiment of a device in accordance with the present invention;

FIG. 7 is a top view of a device in accordance with the anterior box embodiment of FIG. 2B showing the internal mechanism of the assembly box;

FIG. 8 shows exemplary Doppler ultrasound test results obtained by the application of the present invention.

FIG. 9 is a pictorial illustration of the another device fastened to a sleeve placed on a calf of a sitting person in accordance to another embodiment of the present invention;

FIG. 10 is a side view of a sleeve placed on a calf of a sitting person used for fastening a device according to some embodiments of the present invention

FIG. 11 is a schematic illustration of a device with straps for fastening the device according to an exemplary embodiment of the present invention;

FIG. 12 is a schematic illustration of a device according to the present invention fastened to a bandage;

FIG. 13 is a schematic illustration of a bandage used to fasten a device according to an embodiment of the present invention;

FIGS. 14 and 15 are schematic illustrations of a bandage with a tightening strap in accordance to an exemplary embodiment of the invention;

FIGS. 16A and 16B are schematic illustrations of an internal side view and external side view respectively of a sleeve for attaching a circulation enhancement device according to an exemplary embodiment of the invention;

FIG. 17 is a schematic illustration of deployment of a sleeve on a user's limb according to an exemplary embodiment of the invention;

FIGS. 18A and 18B are schematic illustrations of an internal side view and external side view respectively of a sleeve for attaching a circulation enhancement device according to an exemplary embodiment of the invention;

FIGS. 19A and 19B are schematic illustrations of an internal side view and external side view respectively of a sleeve for attaching a circulation enhancement device according to an exemplary embodiment of the invention;

FIG. 20A is a schematic illustration of an alternative sleeve device according to an exemplary embodiment of the invention;

FIG. 20B is a schematic illustration of deployment of a sleeve according to an exemplary embodiment of the invention;

FIG. 21A is a schematic illustration of a sleeve with additional support according to an exemplary embodiment of the invention;

FIG. 21B is a schematic illustration of deployment of a sleeve with additional support according to an exemplary embodiment of the invention;

FIG. 22 is a schematic illustration of a sleeve with protectors for a users joint according to an exemplary embodiment of the invention; and

FIG. 23 is a schematic illustration of deployment of a sleeve with additional support according to an exemplary embodiment of the invention.

### DETAILED DESCRIPTION

A device for the intermittent compression of the extremities muscles for the enhancement of blood and lymph flow in a limb and the prevention of stasis related disorders is disclosed.

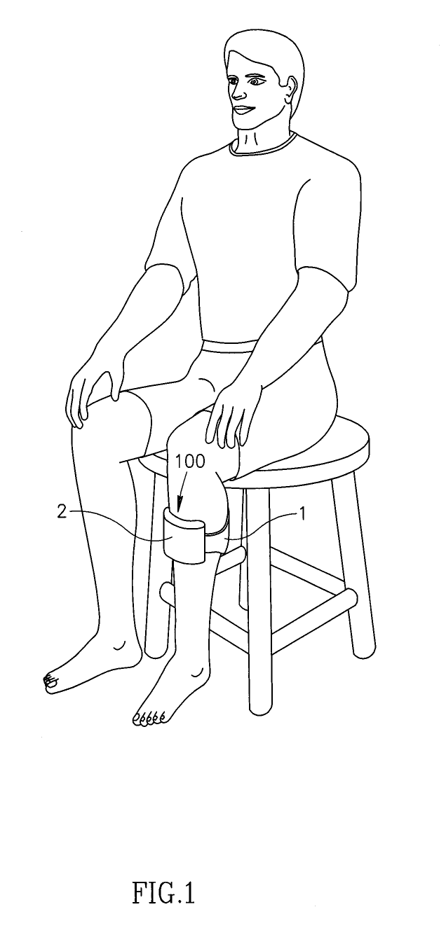

The portable device of the present invention, generally designated 100, is shown in FIG. 1, worn on the calf of a sitting person, Device 100 can be worn directly on the bare limb, or on a garment, such as trousers, worn by the person using the device.

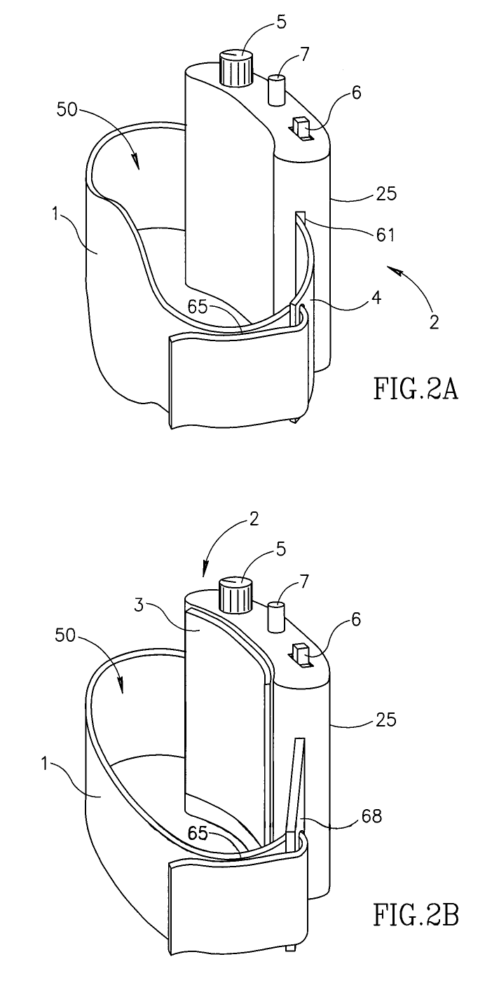

Device 100 comprises two main components, an assembly box 2 which contains all the machinery parts responsible for the device operation, and a strap 1 connected to said assembly box such as to form a closed loop (designated 50, see FIG. 2) for encircling a person limb. The power supply for the device may be of the internal power supply type such as a rechargeable or non rechargeable low voltage DC batteries or an external power supply type is such as an external power outlet connected via an AC/DC transformer such as a 3-12V 1 Amp transformer, fed through electrical wires to a receptacle socket in the device (not shown).

As shown in FIG. 1, strap 1 is preferably wide in the middle and narrow at the ends where it connects to assembly box 2. Strap 1 however may assume any other shape and form such as a constant width belt. The strap can be fabricated from any soft material that is non-irritating to the skin, such as thin plastic, woven fabric and the like. Strap 1 can be fabricated from one material or alternatively can combine more than one material. For example, strap 1 can be made of both non stretchable material and stretchable material wherein such an arrangement may be comprised from a stretchable material for example rubber fabric in the center of the strap 1 and a non stretchable material such as plastic flanking the stretchable material and comprising the rest of the strap. Such an arrangement facilitates more uniform stretch forces on the strap as well as preventing the slippage of the strap from the limb.

According to the preferred embodiment shown in FIG. 1, hereinafter called the anterior box embodiment, strap 1 is placed against the muscles while assembly box 2 is placed against the calf bone. However, according to another embodiment of the present invention, hereinafter called the posterior box embodiment, assembly box 2 can be placed against the muscles. Additionally, assembly box 2 can be positioned with other rotation angles around the muscle.

FIG. 2 illustrate two possible embodiments of the device of the present invention. FIG. 2A represents a preferred embodiment of the present device, in which squeezing the limb muscles for promoting the increase of blood and lymph flow in the limb, is performed by pulling and releasing strap 1, thus, intermittently shortening the effective length of loop 50 encircling the limb. This embodiment is preferably used as an anterior box embodiment of the present invention. However, it will be easily appreciated that the device of FIG. 2A can be used as a posterior box embodiment as well.

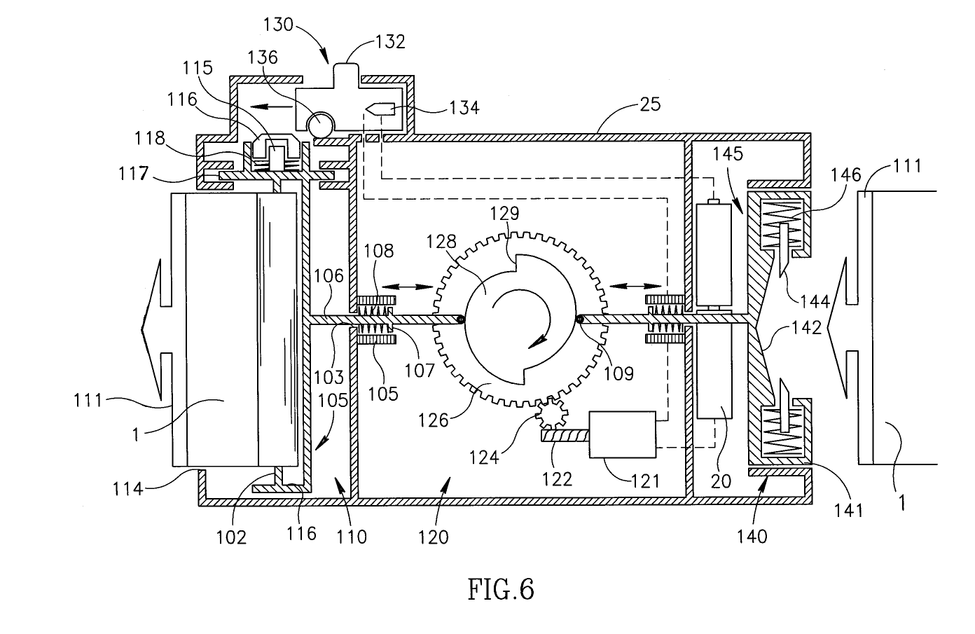

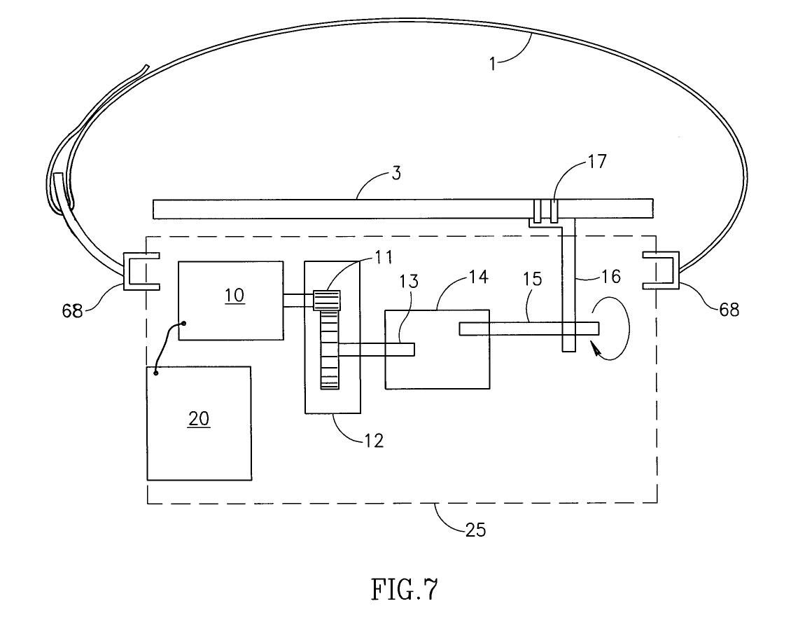

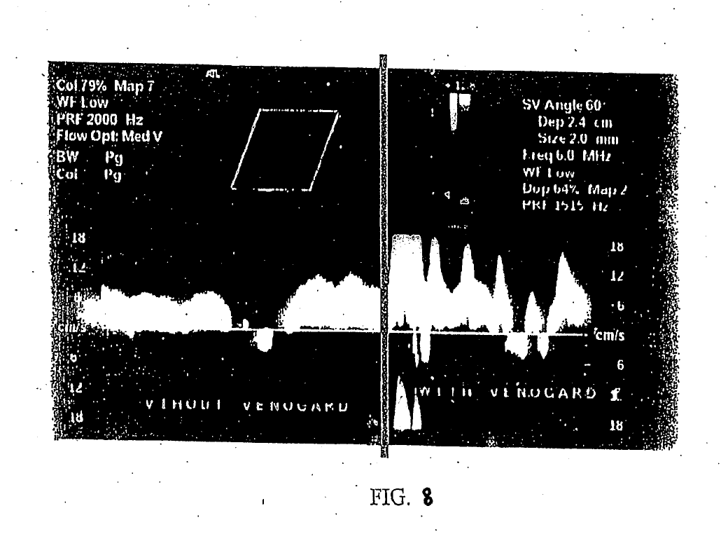

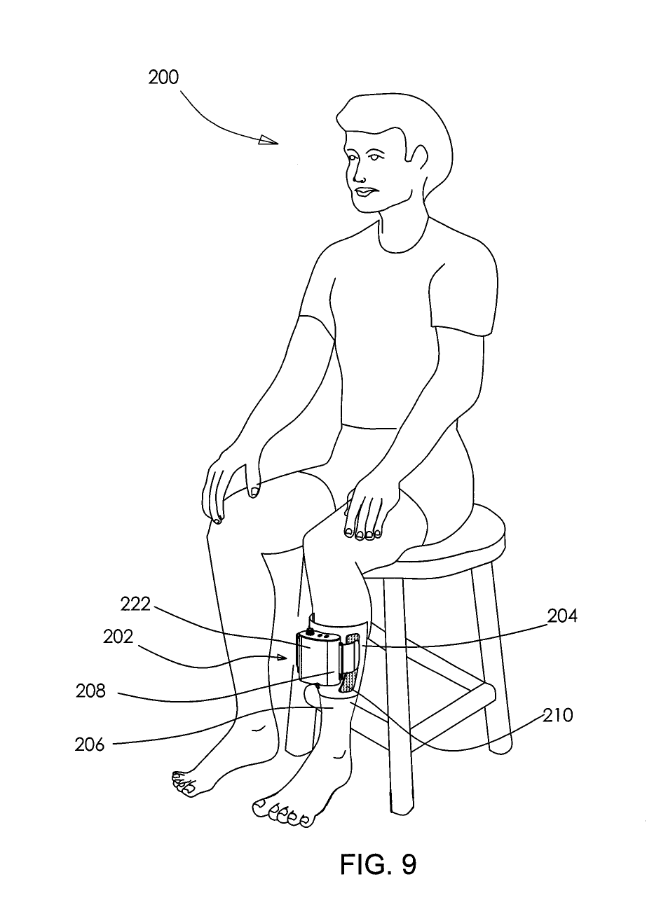

FIG. 2B presents another embodiment of the present device in which assembly box 2 is the active intermittent compressing part by means of mobile plate 3 attached to the box. This embodiment, which can be used only as a posterior box embodiment, will be explained in conjunction with FIG. 6.

Turning back to FIG. 2A, assembly box 2 comprises a thin, curved flask-shaped casing 25 which contains all the parts of internal machinery responsible for intermittent pulling and releasing strap 1. Casing 25 is preferably fabricated from, but not limited to, a plastic molding, a light metal, or any other material which is light, non irritating to the skin, and cheep to produce.

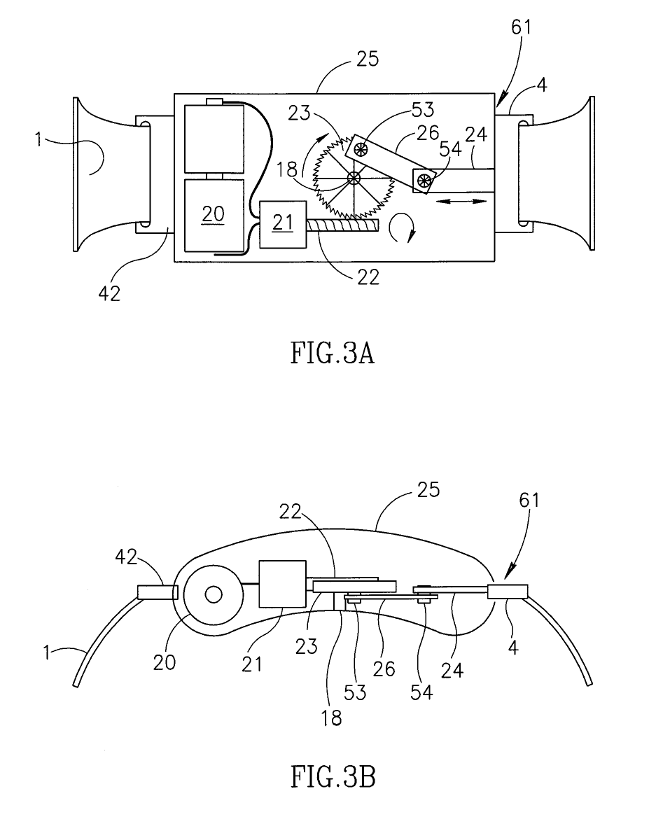

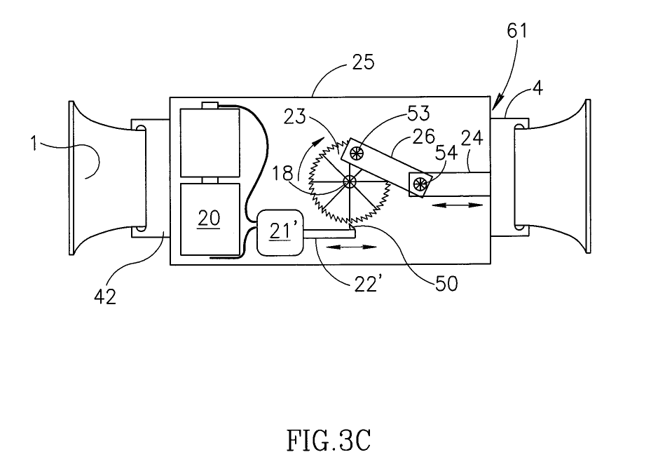

Strap 1 is connected at both its ends to assembly box 2 by means of two buckles 4 and 42 at the sides of casing 25 (buckle 42 not shown). At least one of said buckles (here buckle 4) is a mobile buckle, which can move in and out of casing 25 through slit (opening) 61, thus pulling and relaxing strap 1 between a retracted and a relaxed positions. The retraction protraction motion shortens and lengthens the effective length of strap 1, thus causing intermittent compression of the underlying muscle and increasing the blood and lymph flow in the underlying vessels. Possible inner machinery responsible for activating the intermittent pulling of strap 1 is described in the following in conjunction with FIGS. 3 to 6.

Strap 1 can be adjusted to fit the size of the limb, on which device 100 is to be operated, by having at least one of its ends free to move through its corresponding buckle, such that the strap can be pulled by said end for tightening the strap around said limb. Said end is then anchored in the appropriate position. In the example shown here, the strap is folded back on itself and the overlapping areas are fastened to each other by fastening means 65, such as Velcro™ strips, snap fasteners or any other fastening or securing means. Alternatively, said strap end can be secured to casing 25 by fastening means such as Velcro strips, opposite teeth-like protrusions both on casing 25 and on strap 1, and the like.

The second end of strap 1 can be connected to its corresponding is buckle either in a permanent manner by attaching means such as knots or bolts, or can be adjustable in a similar manner to what had been described above, allowing both ends to be pulled and anchored simultaneously for better fitting.

Yet, in accordance with another embodiment of the invention, the strap can be wound around a retracting mechanism positioned at one side of casing 25. The free end of the strap can be provided with a buckle for allowing connection into the opposite side of casing 25 either by one of the aforementioned means described or by means of a quick connector.

Outer casing box 25 also includes an on/off switch 6, a force regulator 5 for regulating the force exerted on the calf muscle by strap 1 and a rate regulator 7 for regulating the frequency of intermittent compressions. Alternatively, force regulator 5 and on/off switch 6 can be combined into one button. Force regulation can be obtained for example by way of controlling the length of the strap interval between retracted and protracted positions. The length interval between contracted and relaxed positions is preferably, but not limited to, 1-50 millimeters. Frequency regulation can be obtained by way of regulating, but not limited to, the speed of the inner machinery.

A person skilled in the art will readily appreciate that the present invention can be used for the enhancement of both arterial and venous blood and lymph flow in a limb (upper and lower). The examples provided in the following discussion serve as an example and should not be construed as a limitation to the application of the preset invention.

Referring now to FIGS. 3A and 3B, there is shown a side view and a top view respectively of first inner machinery for the device of FIG. 2A. The numbers optionally correspond in both drawings. According to this embodiment, one end of strap 1 is connected to assembly box 2 via a fixed fitting 42 by means such as bolts, knots glue, etc. The second end is connected via a movable buckle 4, which traverses slit 61 located at the side of casing 25. Buckle 4 can retract and protract through opening 61, as described above. Movable buckle 4 is connected to the inner machinery by means of attachment to a rigid push/pull rod 24

The inner machinery responsible for the motion of movable buckle 4 is herein described.

Energy source 20 such as low voltage DC batteries, supply electrical energy to an electrical motor 21 such as, but not limited to, a 3-12 V DC motor, via electrical contacts such as wires.

Electric motor 21 converts electric energy into kinetic energy, spinning a spirally grooved (worm) central shaft 22. Shaft 22 is coupled to a (speed reduction) wheel 23, having complementary anti-spiral circumferential grooves or teeth, causing wheel 23 to revolve around its center which is fixed by axis 18 perpendicular to its surface. An elongated connector plate 26 is pivotally jointed at one end to off-center point 53 on wheel 23 and at its second end to rod 24 at point 54, such that the rotation of wheel 23 actuates plate 26 to intermittently push and pull rod 24, in a crankshaft manner. Consequently, mobile buckle 4 is intermittently pulled inward and outward casing 25 through slit 61, thus intermittently shortening the circumference of loop 50.

A modified machinery, represented in FIG. 3C, includes the following changes with reference to FIGS. 3A and 3B. The electric motor 21 and spinning worm shaft 22 are replaced with an electromagnetic motor 21′ (such as a push-pull solenoid 191C distributed by Shindengen electric Ltd.) having a reciprocating central rod 22′ with an upwardly inclined spike-tooth projection 50 at its end. Rod 22′, via projection 50 is coupled to wheel 23, having complementary teeth. As reciprocating rod 22′ slightly protrudes from, and retracts into the motor body, projection 50 latches sequential teeth of wheel 23 as it protrudes and pulls wheel 23 as it retracts, causing wheel 23 to revolve around its axis. The mechanism of FIG. 3C generates a large force output while minimizing the power input. Such a machinery is very cost effective.

The above description clearly shows how the internal mechanical machinery of the proposed device acts to intermittently shorten loop 50, culminating in intermittent compression of the leg or hand muscle and leading to increase of venous return and helping in the prevention of the formation of deep vein thrombosis.

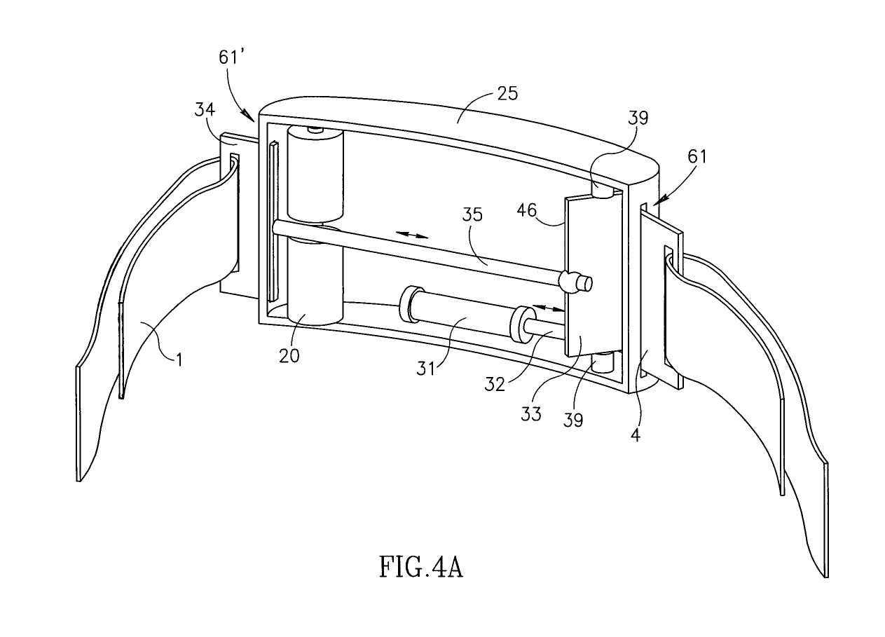

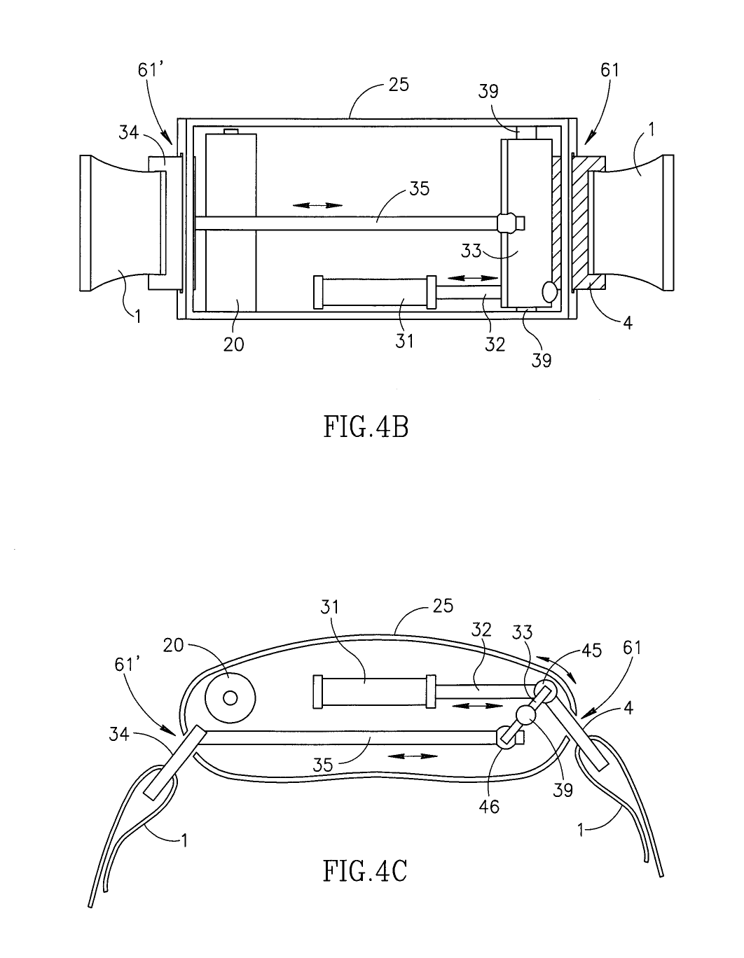

An alternative machinery embodiment for the device embodiment of FIG. 2A is shown in FIGS. 4A, 4B and 4C. FIG. 4A is a perspective drawing view showing the internal parts of assembly box 2 with the frontal part of casing 25 removed. FIGS. 4B and 4C side and top view, respectively of the embodiment shown in FIG. 4A. According to this embodiment, both ends of strap 1 are connected to the inner machinery of assembly box 2 by means of two movable buckles 4 and 34, which can move inwardly and outwardly casing 25 through slits 61 and 61′, respectively.

This alternative embodiment combines the following elements:

A rectangular plate 33 positioned close to one side wall of casing 25, adjacent to slit 61. Plate 33 having two parallel rectangular surfaces, two narrow vertical edges, designated 45 and 46, and two narrow horizontal edges. Plate 33 is pivotally mounted at its narrow horizontal edges to the top and bottom walls of casing 25, by pivoting means 39, such as to allow rotational movement of the plate around the vertical axis connecting between pivoting means 39;

A push-pull electromagnetic motor 31 (such as pull tubular solenoid 190 distributed by Shindengen electric Ltd.) connected via its reciprocating central rod 32 to one vertical edge (45) of the centrally hinged rectangular plate 33, at about mid point of said edge;

A longitudinal rod 35 spans the length of casing 25. Said longitudinal rod 35 is connected at one end to the opposite vertical edge (46) of plate 33 and at its second end to movable buckle 34 positioned at the other side of casing 25.

Centrally hinged rectangular plate 33 is thus connected on one side to the electromagnetic motor 31 via central rod 32, and on the other side to longitudinal rod 35 (as best seen in FIG. 4C). Movable buckle 4 is also connected to narrow edge 45 of plate 33 but extends outwardly, through slit 61, in a different direction than rods 32 and 35.

As can be best seen in FIG. 4C, the reciprocating movement of rod 32 causes plate 33 to turn back and forth around its central axis, preferably the angular displacement is in the range of 20 to 60 degrees. Consequently, buckles 4 (coupled directly to plate 33) and 34 (by means of connecting rod 35) are synchronously pulled and pushed inward and outward of casing 25, resulting in intermittent shortening of the limb encircling loop.

This embodiment is advantageous because the longitudinal rod 35 allows both buckles 34 and 4 to reciprocate each other simultaneously, thus enhancing the efficiency of the device (by enhancing the reciprocating displacement of electromagnetic motor 31) and requiring less energy.

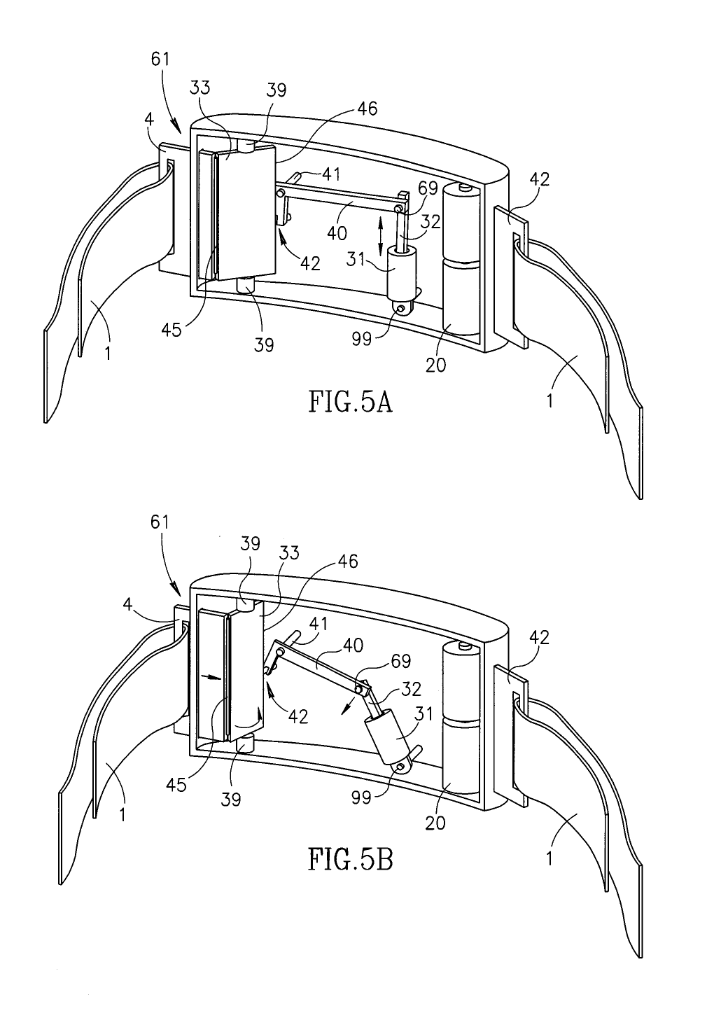

FIGS. 5A and 5B illustrate yet another alternative machinery for the device embodiment of FIG. 2A. The embodiment of FIG. 5 also uses a pull-push electromagnetic motor as the driving force but allows force enhancement by the addition of an “L” shaped lever bar 40 to centrally displaced rod 32 of the embodiment shown in FIG. 4. According to this embodiment, one edge of strap 1 is connected to fixed buckle 42 while the second end is connected to movable buckle 4 which transverse casing 25 through side slit 61. The movable buckle 4 is connected to centrally hinged rectangular plate 33 in a similar manner to what has been described in conjunction with FIG. 4.

In accordance with the present embodiment, electromagnetic motor 32 is pivotally mounted at its rear end to the base by pivoting means 99. The “L” shaped lever bar 40 pivotally mounted at its longer arm end to reciprocating rod 32 by pivoting means 39, and at its shorter arm end is attached to narrow edge 46 of plate 33, by attaching means 42, in a manner which allows it to slide up and down said edge. Such attaching means can be obtained, for example, by railing means such as a groove engraved along the edge of the short arm of lever 40 and a matching protruding railing extending from narrow edge 46 of plate 33. The right-angled corner of “L” shaped bar 40 is pivotally anchored to casing 25 by means of axis 41 perpendicular to the bar surface.

FIG. 5A represents the “relaxed” mode (i.e., buckle 4 in a protracted position), while FIG. 5B is in a “contracted” mode (buckle 4 in a retracted position). To understand the action of this embodiment a static description of the “relaxed” mode followed by the “contracted” mode description is herein given.

The “relaxed” mode in FIG. 5A, illustrates the electromagnetic motor 32 at a perpendicular position to the base of casing 25, and “L” shaped lever 41 in a perpendicularly positioned to reciprocating rod 32.

The “contracted” mode is shown in FIG. 5B. When reciprocating rod 32 retracts into electromagnetic motor 31, it causes the “L” shaped to rotate around axis 41, such that connection 69 moves toward electromagnetic motor 31 as well as toward the rectangular plate 33. This rotation is allowed due to pivot attachment 99 of electromagnetic motor 31 and pivot attachment 41 of “L” shaped lever bar 40. The other end of the “L” shaped lever bar 41 slides in the upward direction on edge 46 of rectangular plate 33 and at the same time it pushes plate 33 causing it to rotate counterclockwise such that edge 45 and consequently buckle 4 are drawn deeper into casing 25.

When reciprocating rod 32 reciprocates its motion, “L” shaped bar 41 returns to its “relaxed” perpendicular position (FIG. 5A) and consequently edge 45, along with buckle 4 are pushed outwardly.

Thus, this chain of events leads to an effective intermittent shortening of the limb encircling loop (50) and to an intermittent compression of the underlying muscle enhancing the blood flow.

FIG. 6 illustrates yet another preferred embodiment of the present invention, including means for allowing asymmetrical contraction-relaxation cycle and in particular for allowing fast contractions, followed by much longer periods of relaxation. Such a cyclic pattern is found to have the most beneficial effect for enhancing blood and lymph flow. In accordance with this embodiment, the machinery components responsible for intermittent pulling and releasing strap 1 comprises a motor 121 having a worm shaft 122, a speed reducing gear comprising wheels 124 and 126, coupled to shaft 122, and a disk 128 of irregular perimeter, concentrically mounted on wheel 126. Double-tooth disk 128 is shaped as two identical halves of varying curvature radius, each having a gradual slope at one end and a cusp 129 where the radius changes abruptly from maximum to minimum at its second end, wherein between two ends the radius of curvature is almost constant. The machinery components, including motor and wheels, are accommodated in a central compartment 120 of casing 25.

Two side compartments, 110 and 140, accommodate laterally movable strap connectors 105 and 145, respectively. Compartments 110 and 140 are provided with side slits 114 and 141, through which strap 1 can slide in and out. In accordance with the embodiment shown here, strap 1 is retractably mounted at one side of casing 25 (compartment 110) and having its free end provided with a quick male connector for connecting into complementary female connector in compartment 140. This strap fastening arrangement allows for quick and simple adjustment of the strap to the size of the limb and for exerting primary pressure on the muscles. Accordingly, connector 105 includes a vertical rod 102 rotatably mounted between two horizontal beams 116 and 117, allowing rod 102 to revolve around its axis for rolling/unrolling strap 1. Strap 1 is affixed to rod 102 at one end and is wound around the rod. Rod 102, acting as a spool for strap 1, is provided with a retraction mechanism (not shown). The retraction mechanism can be any spring loaded retracting mechanism or any other retraction mechanism known in the art, such as are used with seat belts, measuring tapes and the like. For example, the retraction mechanism can comprise a spiral leaf spring having one end secured to rod 102 so as to present torque on the rod when strap 1 is withdrawn and to cause the strap to roll back once its free end is released. The upper end of rod 102 terminates with head 115 and a cap 116 of a larger diameter mounted on springs 118. The inner surface of cap 116 fits onto outer surface of head 115, such that when cap 115 is pressed downward, it locks head 115, preventing free rotation of rod 102 and consequently preventing strap 1 from being rolled or unrolled. The second free end of strap 1 terminates with buckle 111 which fits into a complementary accepting recess 142 of connector 145 for allowing quick connection into the second side of casing 25. In the example illustrated here, buckle 111 has an arrow shape while connector 145 has a complementary arrow shape recess 142 provided with slanted protrusions 144 mounted on springs 146. When buckle 111 (duplicated on the right side of FIG. 6 for description sake only) is pushed toward recess 142, protrusions 144 are pressed aside, then fall behind the arrow head of buckle 111, locking the buckle.

Movable connectors 105 and 145 are coupled to the machinery components by means of horizontal rods 106, which extend through openings 103 into central compartment 120 and are in contact with disk 128 perimeter. Horizontal rods 106 terminate with bearings 109 which allow the rods to smoothly slide along disk 128 perimeter as the disk revolves around its axis. Thus, the distance between rods 106, and consequently the periodical change of the circumference of the loop encircling the limb, mimics the outline shape of disk 128. In order to maintain constant contact between bearings 109 and disk 128 and to facilitate fast transition between strap relaxed to contracted position, rods 106 are mounted on biasing springs 108 positioned between walls 105 and are provided with plates 107 perpendicular to the rod axis and pressed against springs 108. Thus, springs 108 bias connectors 105 and 145 in the inward direction toward each other. As disk 128 revolves around its axis, springs 108 are compressed by plates 107 in accordance with disk 128 varying radius. When disk 128 rotates to the point where cusps 129 simultaneously face bearing 109, rods 106 momentarily lose contact with disk 128 and the potential energy stored in springs 105 is released, pushing rods 106 inwardly. This causes a sudden inward pulling of strap 1 by both rods 106, leading to sharp squeezing of the limb muscles. It will be easily realized that the length interval between contracted and released states of the limb encircling loop, and hence the squeezing force exerted on the muscles, is directly proportional to the radius change at cusp 129. Following the sudden strap contraction, the rods are gradually pushed outwardly leading to strap relaxed mode which lasts for substantially half a cycle. Hence, one revolution of disk 128 around its axis results in two fast strap contractions. Typically, the transition from relaxed to contacted position takes about 0.5 seconds, the transition from contracted to relaxed position takes about 5 seconds and the relaxed position is maintained for about 50 seconds. However, it will be easily realized that the perimeter of disk 128 can be shaped such as to obtain any desired contraction-relaxation cyclic pattern.

The device is further provided with an on/off switch 130 comprising button head 132, electrical connector 134 made of electric conductive material, and a bottom protrusion 136. When switch 130 is pushed to the left by means of head 132, connector 134 closes the electric circuit (shown in broken line), setting the machinery into action. Simultaneously, protrusion 136 presses cap 116 downward, locking head 115 and preventing rod 102 from turning around its axis, for fixing the available length of strap 1. Button 132 can be further provided with a force regulator for regulating the frequency.

A different embodiment of the present invention in which box assembly 2 is the active intermittent compressing part is depicted in FIG. 2B. According to this embodiment, assembly box 2 further comprises a compressing plate 3 lying substantially parallel to casing 25 at a predetermined distance from its surface. According to this embodiment, the assembly 2, more specifically compressing plate 3 is pressed against the muscle and intermittently extends and retracts from casing 25 thus producing intermittent compression of the calf muscle.

According to this embodiment strap 1 is connected to casing 2 by two fixed slitted latches, such that at least one end of strap 1 is threaded through one of latches 68 and is folded onto itself to allow comfortable fitting, as described in conjunction to FIG. 2B. An on/off switch 6, a power regulator 5 and a rate regulator 7 are located at the top of the device in the same fashion as in FIG. 2B.

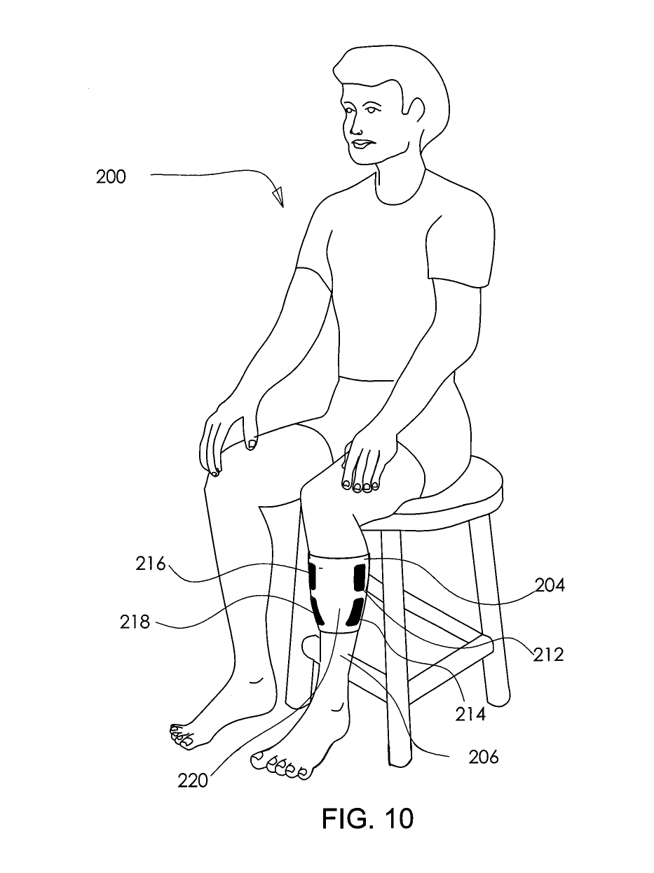

A top view of a machinery embodiment in accordance with the device embodiment of FIG. 2B is shown in FIG. 7. A power source 20 powers an electrical motor 10 that has a centrally located shaft 11. The centrally located shaft 11 is coupled to a velocity reduction gear 12 which reduces the spinning velocity of rod 11 and increases the power output. Reduction gear 12 has a centrally located rod 13 that is connected to drum 14 that has an eccentric located rod 15. The eccentric located rod 15 is connected perpendicularly to the longer arm of a motion transfer L-shaped bar 16, wherein the shorter arm of said L-shaped bar 16 is connected to compressing plate 3 by connection means 17. Connection means 17 may be for example bolts, pins, screws, glue or other means.

Electrical motor 10 converts electrical energy into kinetic energy stored in the spinning of centrally located rod 11. The kinetic energy stored in the spinning of the said centrally located rod 11 is converted into power by velocity reduction gear 12. The power stored in centrally located rod 13 that is connected to velocity reduction gear 12 is converted to rotate drum 14, which is attached to eccentrically located rod 15. The circular motion of eccentrically located rod 15 is transferred to the extension and retraction of compressing plate 3 via motion transfer rod 16 and connection means 17.

According to this arrangement, the circular motion of the eccentrically located rod 15 is transformed to the periodic motion of plate 3. The periodic motion of plate 3 is a combination of a first periodic motion in the extension-retraction direction (i.e., increasing and decreasing the distance between plate 3 and casing 25) as well as a second periodic motion which is perpendicular to said first periodic motion. (In accordance with FIG. 6, this second periodic motion is in a direction perpendicular to the drawing surface).

Thus, further to the obvious effect of applying intermittent is compression on the limb by the extension-retraction motion of plate 3, the present embodiment also imparts the device a “massage-like” effect, thus enhancing the squeezing efficacy.

It will be easily realized by persons skilled in the art that the embodiments described in FIGS. 3-7 are only examples and that different features described separately in conjunction with a particular embodiment, can be combined in the design of a device of the present invention. For example, a retractable strap feature as illustrated in FIG. 6 can be combined with any of the other embodiments. Much the same, an asymmetrical component such as disk 128 of FIG. 6 can be added to any of the other embodiments for allowing a particular pattern of a contraction-relaxation cycle.

FIG. 8 shows an exemplary Doppler ultrasound test result obtained by the application of the present invention. The results shown here were obtained by applying a device in accordance with the embodiment of FIG. 6 on a 49 years old healthy woman in the supine position. The device was applied to the right thigh close to the groin. The right side of FIG. 8 is a Doppler ultrasound measurement of the patient just before the activation of the said device. The white areas represent the blood flow in the deep veins of the thigh. These white areas are taken here as baseline for this subject. The blood flow in the deep veins of the same subject is illustrated in the left picture of FIG. 8 immediately after the device was put into action. FIG. 8 clearly shows the immediate enhancement in the venous blood flow above the baseline upon operation of the device as depicted by higher peaks of white areas. The above Doppler ultrasound example displays the efficiency of the present device.

In addition to the examples shown above, it will be apparent to a person skilled in the art that the device of the present invention can be readily used for the enhancement of blood flow in many situations, for example persons sitting or laying for long periods of time (such as during long air flights, car trips, working long hours in a fixed position or an immobilized person). It will be apparent that it may also be used for the enhancement of blood flow of a patient with diseases such as Diabetes Mellitus and Burger's disease. Also, for the enhancement of lymph flow in the hand of a patient post mastectomy. Other uses not described above will be apparent to the person skilled in the art, wherein the above examples are for the purpose of clarity and should not be regarded as limiting in any way.

FIG. 9 is a pictorial illustration of a device 202 according to the present invention. FIG. 9 illustrates device 202 fastened to a sleeve 204 placed on calf 206 of a sitting person 200. Device 202 is fastened to sleeve 204 with two flexible flaps 208 and 209 (not shown). As shown flap 208 is attached to sleeve 204 by fastening flap 208 to sleeve 204 preferably by means of a loop fastener or other strips having an attaching surface which enables the firm attachment of device 202 to sleeve 204. Device 202 provides periodically, compression and squeezing of a limbs muscles for promoting the increase of blood and lymph flow in the limb. The periodical compression and squeezing of the limbs muscles is performed by a mechanism (not shown) placed within housing 222, for example as described above. Optionally, device 202 provides periodical changes between the contracted and the relaxed states. Flaps 208 and 209 extend from housing 222 of device 202. Flaps 208 and 209 are used to attach and fasten device 202 to sleeve 204. Flaps 208 and 209 are also used to perform periodically squeezing and releasing of calf 206 by pulling and releasing of sleeve 204. Flaps 208 and 209 are flexible, substantially not stretchable and are fabricated from natural materials, synthetic materials or combination thereof. Thus, flaps 208 and 209 can be fabricated from nylon fibers, natural cloth, a combination thereof or other like material. Optionally, the fastening of flaps 208 and 209 is performed by using the hook and loop technology, for example Velcro. The mechanism used for activating device 202 can be any of the mechanisms used within the devices depicted above or within the following applications that are incorporated herewith, Israel patent application serial No. 160185, filed Feb. 2, 2004 titled “A PORTABLE DEVICE FOR THE ENHANCEMENT OF CIRCULATION OF BLOOD AND LYMPH FLOW IN A LIMB”, and Israel patent application serial No. 160214 filed Feb. 4, 2004 titled “A PORTABLE DEVICE FOR THE ENHANCEMENT OF CIRCULATION OF BLOOD AND LYMPH FLOW IN A LIMB”. In some embodiments of the invention, the mechanism of device 202 provides different time periods for different states (i.e. relaxed state and compressed state). The compression and squeezing of the limbs muscles is performed by pulling and releasing of flaps 208 and 209 that are fastened to sleeve 204. Thus, pulling of flaps 208 and 209 fastened to sleeve 204 initializes the compression state. The compression of calf 206 is achieved by device 202 pulling flaps 208 and 209 into housing 222. Flaps 208 and 209 are connected to the mechanism (not shown) within housing 222. Subject to the fact that flaps 208 and 209 are fastened to sleeve 204, pulling of flaps 208 and 209 inwards of housing 222 compresses all the perimeter of calf 206 adjacent to flaps 208 and 209. The manner of fastening device 202 to sleeve 204 and the light weight of device 202 allows person 200 to sit, lie down, stand, walk or even run while device 202 is fastened to sleeve 204. According to the present embodiment a strip of hooks (not shown) are appended to flaps 208 and 209 at the surface fastened to sleeve 204. The strip of hooks attached to flaps 208 and 209 are attached to strips of loops appended to sleeve 204. FIG. 10 presents sleeve 204 placed on calf 206 of a sitting person 200 without device 202 of FIG. 9. Sleeve 204 preferably comprises, loop strips 212, 214, 216 and 218 as well as open surface 220. Loop strips 212, 214, 216 and 218 provide attaching surfaces for flaps 208 and 209 (not shown) of device 222 depicted in view of FIG. 9. Strips 212 and 216 and, strips 214 and 218 are positioned opposite one another, respectively. Loop strips 212, 214, 216 and 218 can be appended to sleeve 204 by an adhesive, for example Super Glue Kexing 502 Puissant Instant Adhesive manufactured by Yuyao Kexing Adhesive Co., Ltd. from the Republic of China. According to other embodiments loop strips 212, 214, 216 and 218 can be sewed to sleeve 204 or prefabricated. According to other embodiments loop strips can be positioned on flaps 208, 209 and hook strips can be positioned on sleeve 204. Alternatively, the loop strip can be any material which can be fastened to the flaps 208, 209 and allow the contraction of the sleeve surface 220 via the operation of the device 222 and the movement of flaps 208, 209. Surface 220 is provided to place housing of a device according to the present invention. Thus, housing 222 of device 202 of FIG. 9 is placed adjacent to surface 220. The presence of strips 212, 214, 216 and 218 are provided to position device 202 at the most suitable location and fasten device 222 to sleeve 204 by placing flaps 208 and 209 on the desired strips of sleeve 204. Furthermore, the fastening method (e.g. Velcro) of device 202 to sleeve 204 provides that device 202 can be removed easily and be repositioned later in another position. Device 202 and encasing sleeve 204 provide the ease of deploying and removing of device 202 from sleeve 204. The term sleeve is used in the context of the present invention interchangeably with the term bandage. The sleeve can be a piece of fabric or cloth surrounding the limb, comprising one or more pieces or one or more straps of fabric or cloth. The sleeve can have one or more layers. One layer of the sleeve can surround the limb and provide an effective sleeve to be used in association with the present invention. One or more types of fabric or cloth can be used in combination with a single sleeve. Alternatively, the sleeve can be a prefabricated piece of fabric or cloth. The sleeve can comprise on one edge and along said edge, a section to which adhesive is applied. Said section can be a small strip of about 1-4 centimeters extending inwardly from the edge of the sleeve substantially along the length of the sleeve. Said section to which adhesive is applied to can be used to attach one side of the sleeve to the other when placed on the limb of person 200. A covering layer can be applied to the adhesive to be removed before use. Said covering can be made from a polymer or a plastic like material, which allows the easy removal prior to applying the sleeve to the limb and attaching one sleeve edge to the other sleeve edge. One such closure can be the loop fastener by Velcro. The sleeve can be reusable or for a single use. Persons skilled in the art will appreciate that the sleeve is used to firmly hold the device 202 such that the extending and retracting of the flaps contracts or expend the sleeve and therefore pressure is applied to the limb of the patient. The sleeve is therefore a means for squeezing the limb. The sleeve can be preferably made of non-woven material. Many non-woven materials can be used in connection with the present invention. Two preferred non-limiting example of a non-woven materials which can be used in association with the present invention are the NJ-050V7S3/L style NewJet material by Nuova F.N.T., Padua, Italy, or the Spunbond 1203420 manufactured by Avgol, Barkan, Israel. The non-woven material preferably comprises viscose fibers, but other fibers can also be used to obtain the same characteristics of the non-woven materials allowing the squeezing of the limb as a result of contracting the flaps.

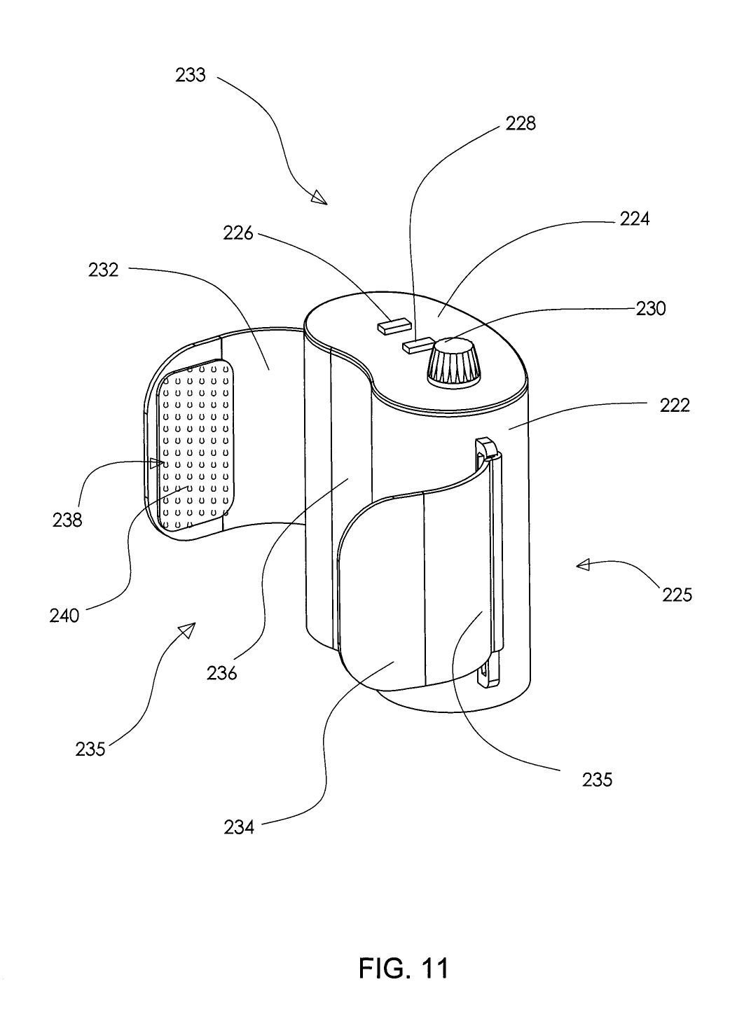

FIG. 11 is an isometric view of a device 233 according to another preferred embodiment of the present invention. Device 233 comprises an assembly box 225 and flaps 232 and 234. Assembly box 225 comprises casing 222 that further comprises an on/off switch 226, a force regulator 228 for regulating the force exerted on the calf muscle by flaps 232, 234 and a rate regulator 230. Switch 226, force regulator 228 and rate regulator 230 are placed on side surface 224 of casing 222. Casing 222 comprises also curving 236 that enables comfortably positioning of casing 222 against a limb encased with a sleeve such as described above. Thus, assembly box 225 can be placed against a calf bone of a limb. Flaps 232, 234 partially encircle the limb's circumference, thus said flaps are placed against the muscles. Hence, device 233 comprises with assembly box 225 and flaps 232 and 234 a partially open loop 235. According to other embodiments of the present invention assembly box 225 can be placed against the muscles correlating to the posterior embodiment discussed in view of FIGS. 1 and 2 above. Casing 222 further encases a mechanism (not shown) and a power source (not shown) that both enable device 233 to perform periodically squeezing of muscles for promoting the increase of blood and lymph flow in the limb. The mechanism with casing 222 can be any of the mechanism depicted above in view of FIG. 9 above. The squeezing of muscles performed by device 233 is performed by applying a movement of one or both flaps 232, 234 in and out of casing 222 by the mechanism within casing 222. Flaps 232 and 234 comprise a strip of hooks 238 and 239 (not shown), respectively. Strips of hooks 238 and 239 are positioned in the part of flaps 232 and 234 that are adjacent to sleeve 204 placed on the limb of person 200 as depicted in FIG. 9. Strips 238 and 239 comprise hooks 240 that can be fastened to strips of loops appended to sleeve 204 as shown in FIGS. 9 and 10 above. Thus, strips 238 and 239 are attached to strips of loops 212, 214, 216, 218 positioned on sleeve 204 as depicted in FIGS. 9 and 10. The movement of one or both flaps 232 and 234 provides periodic squeezing and relaxation of the muscle by stretching and releasing of sleeve 204. The mechanism and the power source used for generating the movement of flap 232 and 234 can be any of the mechanisms and power sources described above. Optionally, the edges 235 and 237 (not shown) of flaps 232 and 234, are connected to the mechanism in casing 222. The portion of flaps 232 and 234 connected to the mechanism in casing 222 can be very small and may range between 0.5-5 centimeters. The movement of flaps 232 and 234 in and out of casing 222 provides that a small portion of the flaps are inserted in casing 222 during the squeezing state and similarly a small portion is pushed out or released out of casing 222 during the relaxation state. Thus, the change of the exposed flaps 232 and 234 adjacent to the sleeve provides intermitted squeezing of the muscles of the limb. Alternatively, one edge 235 or 237 is connected to any of mechanism depicted above while the other edge is fixed to casing 222, thus, providing that only one flap pulls and stretches sleeve 204.

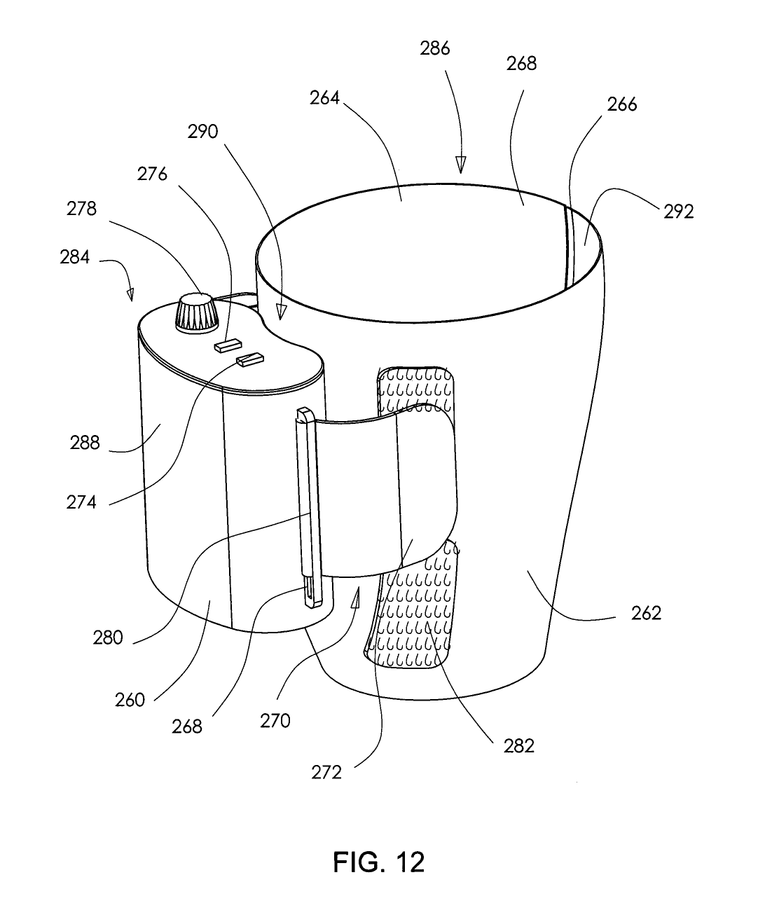

FIG. 12 is a close-up external isometric view of a device 284 according to an embodiment of the invention. Device 284 is fastened to a bandage 262. Bandage 262 can be placed on limb such as a calf of a person. Device 284 can be similar to devices 202 and 233 depicted in view of FIGS. 9 and 11, above. Device 284 comprises an assembly box 260, flaps 270 and 271 (not shown). Assembly box 260 comprises a casing 288 that further comprises an on/off switch 274, a force regulator 276 for regulating the force exerted on the calf muscle by flaps 232, 234 and a rate regulator 278. Casing 288 comprises a mechanism such as depicted above in view of FIGS. 9 and 11. The mechanism within casing 288 is connected to buckles 268 and 269 (not shown). The mechanism within casing 288 can pull and release one or two of buckles 268 and 269. Edges 272 and 273 (not shown) of flaps 270 and 271 (not shown), respectively, comprise a strip of Velcro hooks or other fastening elements. Bandage 262 comprises strips of loops 282 and 283 (not shown). Casing 288 comprises a concave surface that is placed against surface 290 of bandage 262 on a calf of a person. Surface 290 is located between adjacent strips 282 and 283. Device 284 is fastened to bandage 262 by placing casing 288 adjacent to surface 290 and pressing edges 272 and 273 (not shown) against strips 282 and 283 (not shown), respectively. Optionally, fastening device 284 to bandage 262 uses hook and loop fasteners such as Velcro to attach device 284 to bandage 262. Buckles 268 and 269 pulling flaps 270 and 271 (not shown) squeeze the muscles adjacent to device 284 around the perimeter of the persons calf surrounded by bandage 262. The squeezing of the muscle is achieved by the pulling and stretching of bandage 262 at strips 282 and 283 by flaps 270 and 271 and strips 272 and 273. Similarly, the relaxation state is achieved by releasing of buckles 268 and 269. According to the present invention bandage 262 can replace or overlap the sleeve depicted in FIGS. 9 and 10. Alternatively, bandage 262 can be put over a bare calf of a person or over a person wearing trousers. Bandage 262 can be fabricated from woven synthetic material, natural material or combination thereof. Thus, bandage 262 can be woven from nylon fibers. Bandage 262 is an easily handled, flexible and has a length larger than the perimeter of calf of a person (not shown). Bandage 262 in its open state (not shown) is flat. The open state of bandage 262 is first placed on a calf of person and than encases the calf by closing the bandage as depicted below. The place on the calf of a person is chosen according to the location that will provide the most effective results for preventing stasis related disorders by fastening a device 284 or other devices according to the present invention. After placing bandage 262 in the open state on the persons calf, bandage 262 is wrapped around the perimeter of the calf. Wrapping bandage 262 around the calf forms a loop 264. Loop 264 is created by one end (not shown) of bandage 262 that overlaps part of second end 286 of bandage 262 forming an overlapping line 266. Loop 264 remains closed by fastening (e.g. with Velcro) the second overlapping end 286 of bandage 262 with the side not facing the calf of end 286. Thus, end 286 comprises on the side facing out of loop 264 a strip of loops (not shown) in addition to strips 282 and 283 (not shown) that are adjacent to surface 290. Overlapping end 286 that extends from edge 292 and comprises a strip of hooks (not shown) facing inwards of loop 264. Accordingly, overlapping end is fastened to end 286 with Velcro and bandage 262 firmly encases the persons calf. All strips of hooks and loops attached to flaps 270 and 271 (not shown) and to bandage 262 are connected by an adhesive such as Super Glue, sewn to bandage 262 or appended in another manner.

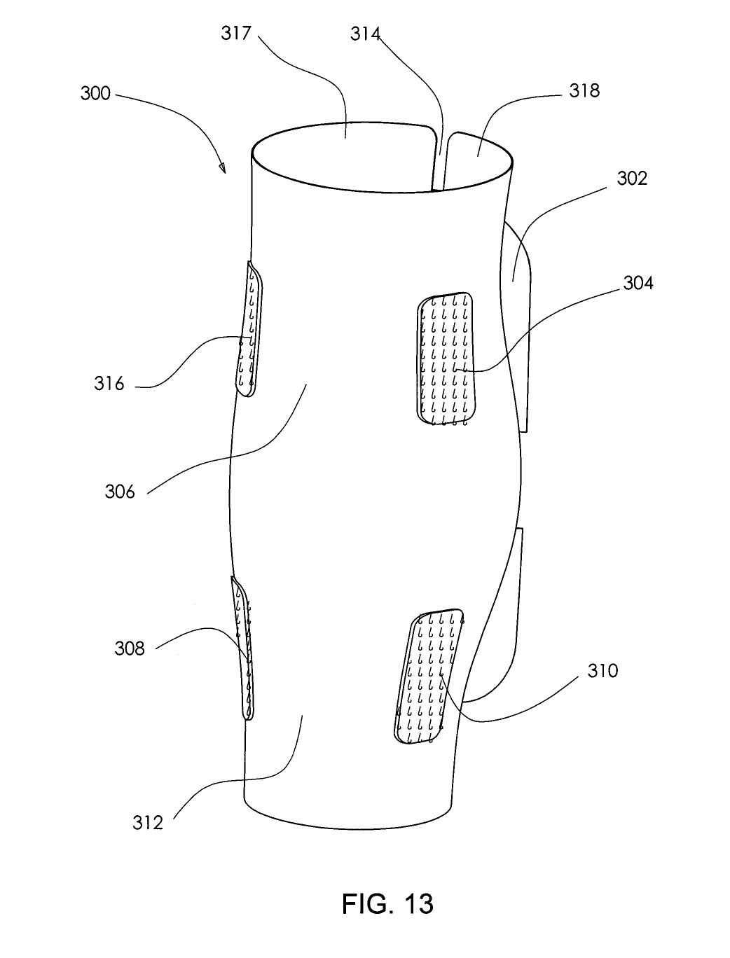

FIG. 13 is an isometric view of a bandage 300 used to fasten devices such as depicted in view of FIGS. 9, 11 and 12 above. Bandage 300 is flexible and comprises a loop 317 that encases a calf of a person (not shown). Bandage 300 comprises four strips of loops 316, 308, 304 and 310 that can be used for fastening a device such as depicted in view of FIG. 12 above (e.g. Velcro).

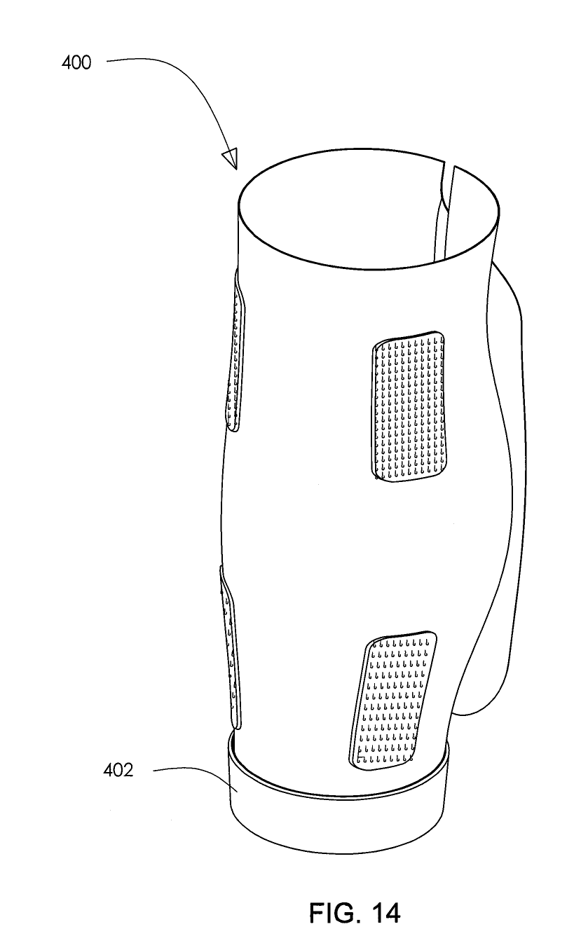

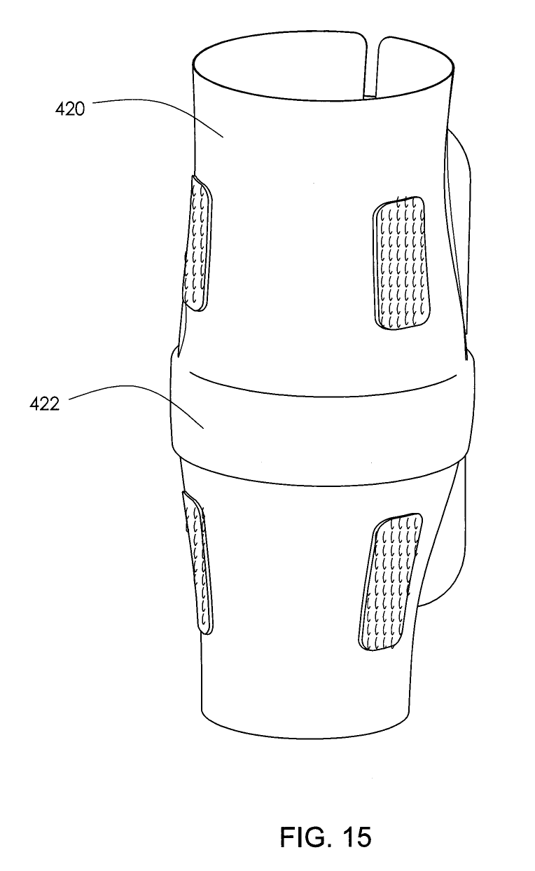

Similar to bandage 262, bandage 300 can be fabricated from woven synthetic material, natural material or combination thereof. Bandage 300 is semi-rigid and can firmly encase a person's calf. Hence, bandage 300 is substantially not stretchable or is semi-rigid. The materials Similar to bandage 262 of FIG. 12, bandage 300 in its open state (not shown) comprises two ends 302 and 318. End 302 overlaps end 318 and fastens bandage 300 with Velcro. Accordingly, end 302 comprises a strip of hooks (not shown) adjacent to the edge of end 302 placed on the side facing the persons calf, and end 318 comprises a strip of loops (not shown) adjacent to an edge of end 318 facing outside of the persons calf. Space 314 presents the state of bandage 300 prior to creating loop 317 by fastening end 302 to end 318 with Velcro. Bandage 300 comprises further surfaces 306 and 312. Surfaces 306 and 312 provide a location for placing a casing such as 288 of device 284 depicted in FIG. 12. Thus, bandage 300 provides multiple locations for placing a device according to the present invention. Alternatively, more than one device (not shown) according to the invention can be used on one bandage 300, concurrently. Bandage 300 provides an encasing element for the calf (not shown). Bandage 300 due to its flexible fabricating material and its semi-rigid quality receives while forming loop 317 the shape of the calf (not shown) it is encasing. Thus, bandage 300 envelopes the person's calf. According to the invention device such as depicted in FIGS. 9, 11 and 12 above is fastened to bandage 300 placed on calf (not shown). After activating device (not shown) fastened to bandage 300 the flaps as depicted above are pulled and released by the device according to a pre-designated time intervals and provide intermittent squeezing forces on the limb by pulling bandage 300. According to other embodiments, bandages fabricated from other materials do not have semi-rigid quality. Thus, FIGS. 14 and 15 present bandage 400 and 420 that require tightening straps 402 and 422, respectively. Bandage 400 and 420 from FIGS. 14 and 15 are fabricated from natural, synthetic or combination thereof materials. Opposed to the sleeve depicted in FIGS. 9 and 10 and bandages depicted in FIGS. 12 and 13, bandages 400 and 420 require tightening straps 402 and 422, respectively, to place bandages 400 and 420 juxtaposed to a calf (not shown). Aside from the fabricating materials, bandages 400 and 420 are optionally, identical to bandage 300 by comprising each four strips of loops having between surfaces providing sufficient place for locating a device as depicted in FIG. 12. Additionally, bandages 400 and 420 each have two ends that attach with Velcro as depicted regarding to ends 302 and 318 of bandage 300. Tightening straps 402 and 422 are fabricated from rubber, synthetic or natural, that attaches bandages 400 and 402 to calf (not shown). All strips of hooks and loops are appended to bandage 300 by adhesive such as Super Glue and the like; alternatively, the strips can be sewed to bandage 300, 400 and 420. Optionally, Tightening straps 402 and 422 are located on the top, the middle or the bottom of the sleeve.

One skilled in the art can appreciate that the embodiments depicted above are provided for a better comprehending of the invention and that other embodiments reflecting other aspects of the invention can be provided. Thus, other embodiments can be provided with a device having flaps that are longer and/or that overlap a larger perimeter of the calf than shown in the embodiments above. Alternatively a device according to the present invention can be provided with flaps that are as long as the perimeter of a calf or even larger. Thus, upon positioning of device on a calf the flaps comprising Velcro strips on both sides of flaps are fastened to a calf by placing one flap overlapping the other. Furthermore, according to another embodiment one flap can be used that for fastening a device to a sleeve or bandage placed on calf with Velcro. According to the last embodiment casing of the device adjacent to sleeve or bandage can be fastened with Velcro as well. Other embodiments can comprise other means for fastening of device to calf such as a zipper, buttons or flaps having hooks on edges of flaps replacing the Velcro strips of that by placing them within loops positioned on a sleeve or bandage overlapping a calf.

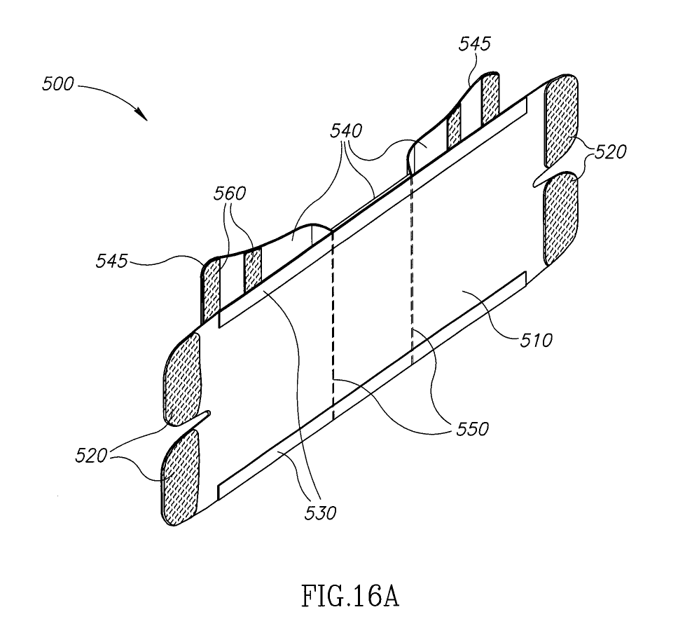

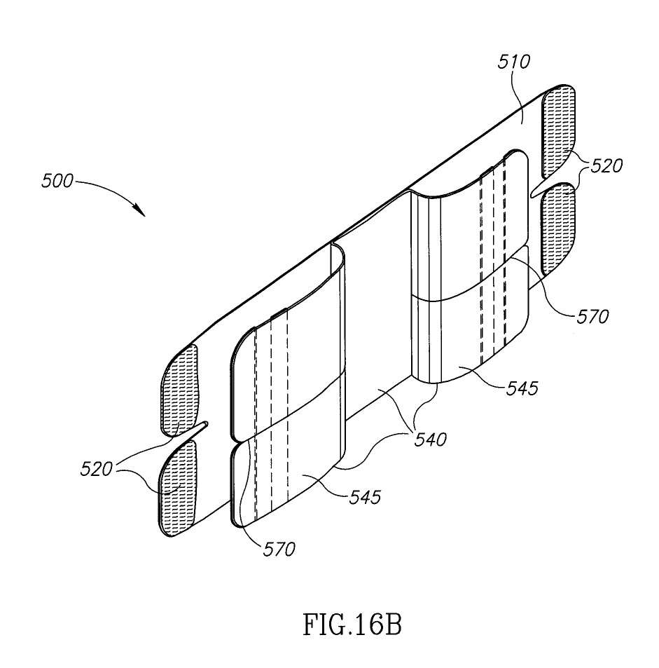

FIGS. 16A and 16B are schematic illustrations of an internal side view and external side view respectively of a sleeve 500 for attaching a circulation enhancement device, according to an exemplary embodiment of the invention. In an exemplary embodiment of the invention, sleeve 500 comprises two layers of material. Optionally, a first inner layer 510 that is used to substantially enclose around a user's limb in order to assure a good grasp that will prevent slippage, and a second outer layer 540 to interface a circulation enhancement device, for example device 233 as shown in FIG. 11. Optionally the two layers are connected together in order to functionally complement each other. Optionally, in use the position of outer layer 540 is limited by the position of inner layer 510.

In some embodiments of the invention, sleeve 500 is manufactured in various sizes to fit different sized users and/or different sized limbs. Optionally, inner layer 510 comprises an elastic material so that it can be stretched around the circumference of different sized limbs. In an exemplary embodiment of the invention, the edge of inner layer 510 is lined with an elastic lining 530 to mark the edge of inner layer 510 or to enhance attachment of the inner layer to the user's limb. In some embodiment of the invention, lining 530 is more elastic than inner layer 510 or inner layer 510 is substantially not elastic.

In an exemplary embodiment of the invention, outer layer 540 comprises a non elastic material so that the pulling forces that will be exerted by the circulation enhancement device will cause pressure on the user's muscles and not just stretch the material.

In an exemplary embodiment of the invention, the ends of inner layer 510 comprise fasteners 520, for example hook and loop fasteners in order to close inner layer 510 over the user's limb. In some embodiments of the invention, one side of fastener 520 is hook material and the other side is loop material in order to enable attachment by matching a fastener 520 from one side of inner layer 510 with a fastener 520 from the other side. Alternatively, fasteners 520 on one side are hook material while fasteners on the other side are loop material. It should be noted that hook and loop fasteners allow flexibility in the exact position of closure of the fasteners, however other types of fasteners may be used, for example snaps or a zipper.

In some embodiments of the invention, each side of inner layer 510 comprises two fasteners 520 to allow more flexibility in closing inner layer 510 over the user's limb, however one fastener, larger or smaller can be used, or more than two fasteners.

In an exemplary embodiment of the invention, outer layer 540 is sewn to inner layer 510 at one or more places along its length to enhance the strength of the connection. Optionally, the sewing forms seam lines 550 which connect between inner layer 510 and outer layer 540. To the right and left of seam lines 550 outer layer 540 is not connected, forming two flaps 545. In some embodiments of the invention, inner layer 510 and outer layer 540 are connected by welding the materials together, for example by using heat or other methods.

In an exemplary embodiment of the invention, the exterior of inner layer 510 is manufactured from hook and loop material, for example loop material. Optionally, along flaps 545 on the side facing the exterior of inner layer 510 are attached stripes of hook material 560 so that flaps 545 will cling to the exterior of inner layer 510 when deployed. Alternatively the inner side of inner layer 540 is manufactured from hook material to cling to the exterior of inner layer 510.

In an exemplary embodiment of the invention, the exterior of outer layer 540 is manufactured from hook and loop material, for example loop material, in order for the flaps of the circulation enhancement device to cling to outer layer 540, for example as shown in FIG. 12.

In some embodiments of the invention, flaps 545 are formed from pieces which are stitched together or separated and reunited instead of a single piece of material in order to enhance flexibility of the flap. FIG. 16B shows seams 570, which unite two parts of flap 545. Optionally, the materials used may be woven to enhance their strength or non-woven to reduce cost.

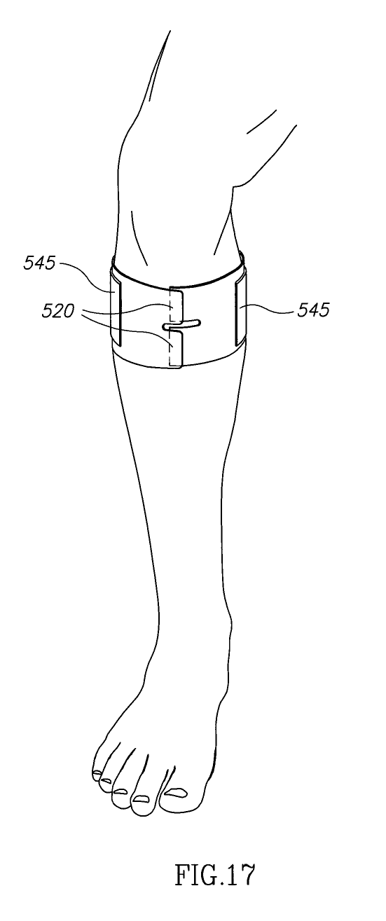

FIG. 17 is a schematic illustration of deployment of sleeve 500 on a user's limb according to an exemplary embodiment of the invention. In an exemplary embodiment of the invention, inner layer 510 as shown in FIG. 16A is placed around a user's limb with flaps 545 on the exterior. Optionally, inner to layer 510 is pulled tight and fasteners 520 are used to secure it in place surrounding a user's limb. In an exemplary embodiment of the invention, flaps 545 are pulled forward toward fasteners 520 and attached on the exterior side of inner layer 510 using fasteners 560. In an exemplary embodiment of the invention, a circulation enhancement device is placed over flaps 545 as shown in FIG. 12.

Optionally, the use of two layers for sleeve 500 allows inner layer 510 to be designed for clinging to a user's limb disregarding considerations for attaching the circulation enhancement device, while outer layer 540 is designed to maximize attachment and application of pressure on a user's limb disregarding considerations regarding clinging to a user's limb.

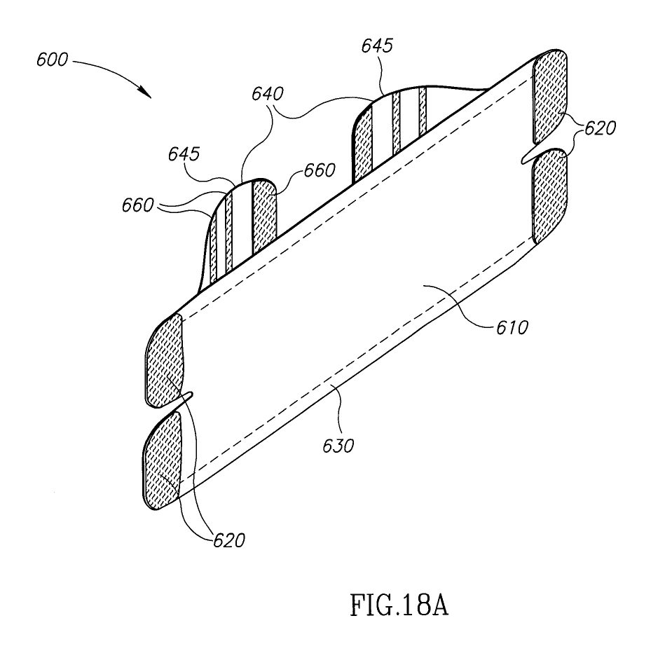

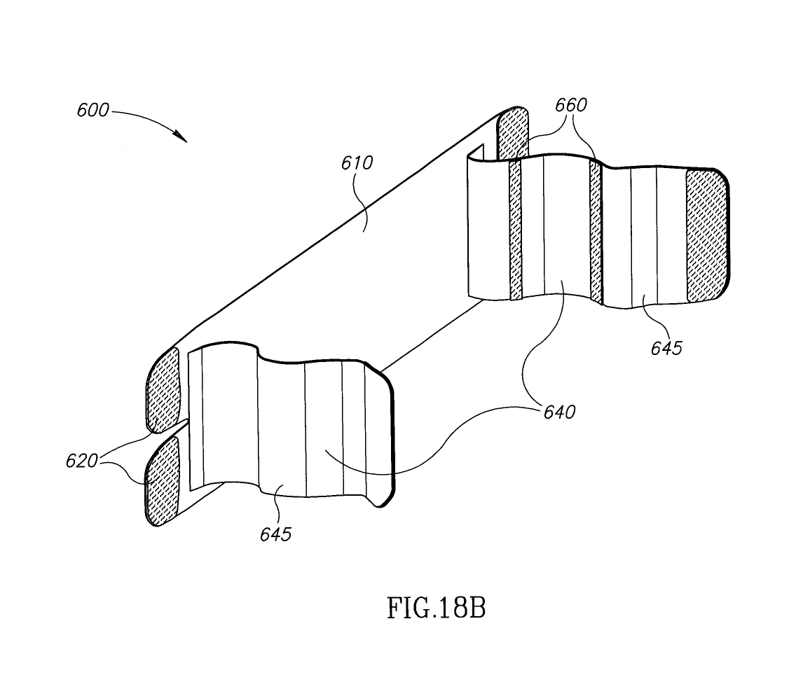

FIGS. 18A and 18B are schematic illustrations of an internal side view and external side view respectively of a sleeve 600 for attaching a circulation enhancement device, according to an exemplary embodiment of the invention. In an exemplary embodiment of the invention, sleeve 600 is analogous to sleeve 500 as shown in FIGS. 16A and 16B. Optionally, sleeve 600 comprises an inner layer 610 and an outer layer 640. Inner layer 610 comprises fasteners 620 at the ends and an elastic liner 630 along the longitudinal edge as in inner layer 510. Optionally, outer layer 640 comprises two flaps 645 and strips 660 as in outer layer 540. However, in contrast to outer layer 540, outer layer 640 is split into two separate flaps 645 and not attached as a continuous piece. In an exemplary embodiment of the invention, flaps 645 of outer layer 640 are connected (e.g. sewn) to inner layer 610 at the ends instead of being connected in the middle of inner layer 610. Optionally, the materials used for sleeve 600 are the same as those used for sleeve 500.

In an exemplary embodiment of the invention, similar to sleeve 500, inner layer 610 of sleeve 600 is placed around a user's limb with flaps 645 on the exterior side. Optionally, inner layer 610 is pulled tight and fasteners 620 are used to secure it in place surrounding the user's limb. In an exemplary embodiment of the invention, in contrast to flaps 545, flaps 645 are pulled backward away from fasteners 620 and attached toward the center of the exterior side of inner layer 610 using fasteners 660. In an exemplary embodiment of the invention, a circulation device is deployed on sleeve 600 over flaps 645 analogous to the illustration in FIG. 12.

Optionally, attaching flaps 645 on the opposite side of fasteners 620 allows adjusting the tightness of flaps 645 while a circulation enhancement device is deployed over flaps 645, in contrast to sleeve 500, which requires removing the device to adjust the tightness of flaps 545.

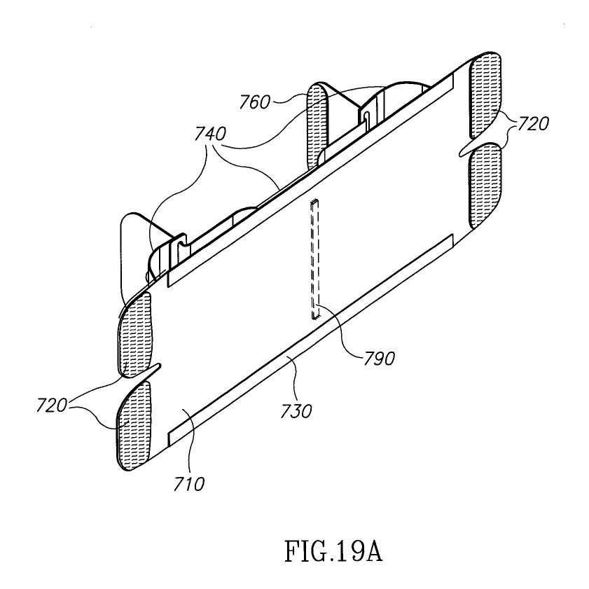

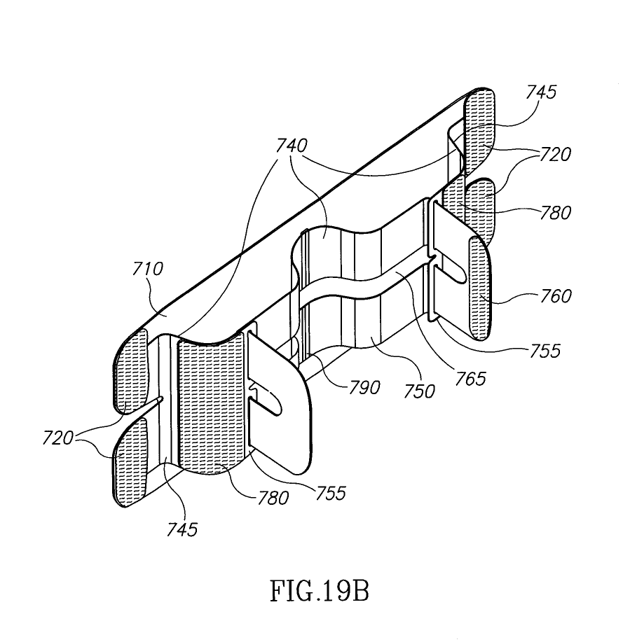

FIGS. 19A and 19B are schematic illustrations of an internal side view and external side view respectively of a sleeve 700 for attaching a circulation enhancement device, according to an exemplary embodiment of the invention.

In an exemplary embodiment of the invention, sleeve 700 is analogous to sleeve 500 and sleeve 600 as shown in FIGS. 16A, 16B, 18A and 18B. Optionally, sleeve 700 comprises an inner layer 710 and an outer layer 740, which is connected to the external side of inner layer 710. Inner layer 710 comprises fasteners 720 at the ends and an elastic liner 730 along the longitudinal edge as in inner layer 510.

In an exemplary embodiment of the invention, outer layer 740 comprises three pieces. The first piece is a strap 750 that is connected in the center to inner layer 710 as in outer layer 540. The second and third pieces are flaps 745, which are connected to the ends of the external side of inner layer 710 as in outer layer 640. Optionally, flaps 745 comprise a buckle 755 through which strap 750 is inserted. In some embodiments of the invention, strap 750 is a solid elongated shape, for example a rectangle or oval or combination of the two. Optionally, buckle 755 comprises a single notch for grasping the ends of strap 750. Alternatively, strap 750 may have its center 765 removed forming a circumference of an elongated shape. Optionally, buckle 755 comprises two notches, one for the upper part of the circumference and one for the lower part in order to prevent strap 750 from disengaging itself from buckle 755.

In an exemplary embodiment of the invention, flaps 745 are comprised from hook and loop material, for example loop material, or the external side of flaps 745 are covered with hook and loop fastener material 780, for example loop material, in order for the flaps of a circulation enhancement device to cling to the external side of flaps 745, for example as shown in FIG. 12.

In an exemplary embodiment of the invention, strap 750 is comprised from hook and loop material, for example loop material, or the external side of strap 750 is covered with hook and loop fastener material, for example loop material, in order to allow one or more strips 760 to attach to the external side of strap 750 when deployed.

In an exemplary embodiment of the invention, strap 750 is sewn at its center to the external side of inner layer 710, forming seams 790. Alternatively the center of strap 750 is connected to the external side of inner layer 710 using other means, for example snaps or, hook and loop fasteners.

In an exemplary embodiment of the invention, the materials used for inner layer 710 and outer layer 740 may be woven to enhance its strength or non-woven to reduce costs. Optionally, inner layer 710 is prepared from an elastic material or at least comprises elastic edges 730 to enhance its grip of the user's limb. In contrast, outer layer 740 is optionally prepared from a non-elastic material to allow exertion of pressure on the user's muscles.

In an exemplary embodiment of the invention, the inner side of inner layer 710 is wrapped around a user's limb. Inner layer 710 is pulled tight and fastened over the limb with fasteners 720 as shown in FIG. 17. In an exemplary embodiment of the invention, the user grasps the ends of strap 750, which are inserted through buckles 755 and pulls them backward toward the back of the user's limb to the opposite side of the closure with fasteners 720. Optionally, the closure of outer layer 740 enables attachment of a device for circulation enhancement with a tight grasp of the user's muscle.

In an exemplary embodiment of the invention, sleeve 700 can be tighten while a circulation device is deployed, in contrast to sleeve 500, which requires removing the device to adjust tightness.

In some embodiments of the invention, sleeves 500, 600 and 700 are degradable so that they need to be replaced after a short period of use. In some embodiments of the invention, sleeves 500, 600 and 700 are replaced for hygienic reasons. Optionally, sleeves 500, 600 and 700 are comprised from cheap material so that they can have a low cost and be considered disposable.

In an exemplary embodiment of the invention, a single sized sleeve (e.g. 500, 600 and 700) can be used by a range of different sized users or different sized limbs, since the sleeve is adjustable. Optionally, different sized sleeves (e.g. small, medium, large or specific sizes) can be supplied to allow for a greater range of adjustment.

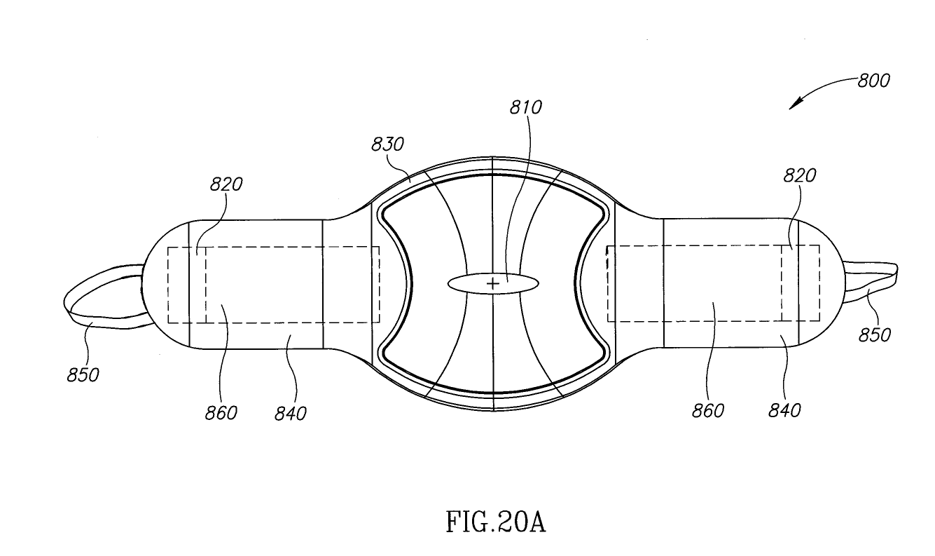

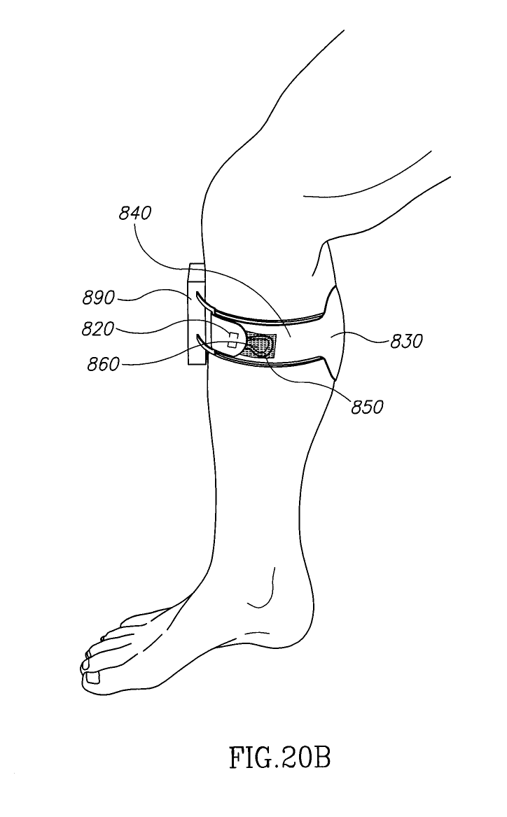

FIG. 20A is a schematic illustration of an alternative sleeve device 800 according to an exemplary embodiment of the invention. In an exemplary embodiment of the invention, sleeve 800 comprises a wider interface 830 to interface a user's limb and a narrower strap 840 to attach it to a circulation enhancement device, for example as shown in FIG. 4A. Optionally, an elastic extension 850 is attached (e.g. by sewing) to both ends of sleeve 800 to assist the user in deploying sleeve 800 around his or her limb. In some embodiments of the invention, extension 850 is formed as a loop. Alternatively, extension 850 is a short piece of elastic extending from the ends of sleeve 800.

In an exemplary embodiment of the invention, the inner side, which is used to interface the user, is manufactured or coated with a biocompatible material so that the sleeve will be comfortable and not irritate the user's skin. Alternatively, other materials may be used including irritating materials since the device may optionally, be deployed over a user's clothes.

In an exemplary embodiment of the invention, interface 830 of sleeve 800 has a piece cut out 810 in order to enhance the grasp of the user by sleeve 800. Optionally, interface 830 is formed as part of a sphere to enhance its grasp of the user's limb. Alternatively, interface 830 is flat.

In an exemplary embodiment of the invention, the external side of sleeve 800 comprises an attachment area 820 and an attachment receiving area 860. Optionally, in deployment attachment area 820 is folded over and attached to attachment receiving area 860, for example attachment area 820 may comprise hook fastener material and attachment receiving area 860 may is comprise loop fastener material. Alternatively other attachment means may be used for, example snaps or adhesives.

In some embodiments of the invention, sleeve 800 is degradable so that it needs to be replaced after a short period of use. Optionally, the adhesion of attachment area 820 wears out over time or the material of sleeve 800 disintegrates and/or tears. Optionally, sleeve 800 is comprised from a cheap material, for example a non-woven material so that it is sold for a cheap price and can be considered disposable. In some embodiments of the invention, sleeve 800 is comprised from a non-elastic material so that it squeezes the user's muscle when pulled by a circulation enhancement device instead of expanding elastically.

In an exemplary embodiment of the invention, a single sized sleeve can be adjusted to be used by a range of different sized users or different sized limbs, for example by attaching attachment area 820 to various positions on attachment receiving area 860. Alternatively, different sized sleeves (e.g. small, medium, large or specific sizes) can be supplied to allow for a grater range of adjustment.

FIG. 20B is a schematic illustration of deployment of sleeve 800 according to an exemplary embodiment of the invention. In an exemplary embodiment of the invention, sleeve 800 is inserted through hooks or buckles on a circulation enhancement device, for example as shown in FIG. 4A. Optionally, attachment area 820 is attached to attachment receiving area 860 in order to secure the circulation enhancement device in position to squeeze a user's muscle.

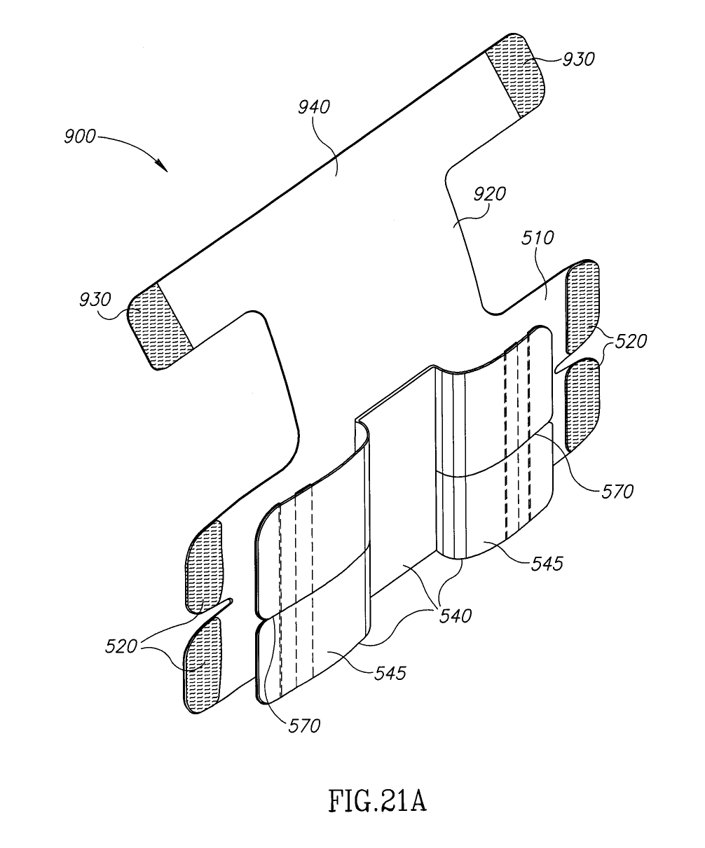

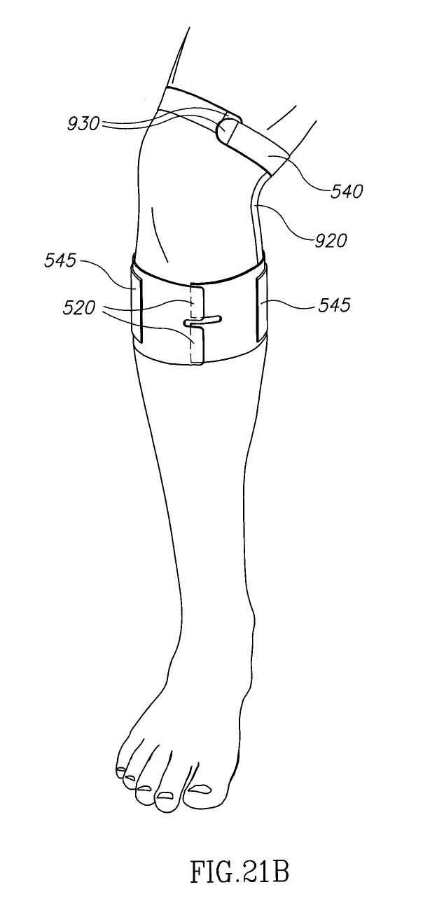

FIG. 21A is a schematic illustration of a sleeve 900 with additional support according to an exemplary embodiment of the invention.

In some embodiments of the invention, sleeve 900 comprises a base sleeve such as described above regarding sleeve 500, 600, 700 or 800, and additionally, a support strap 940 for supporting the base sleeve to prevent it from slipping. Optionally, support strap 940 is attached by an attachment 920 to the base sleeve. In some embodiments of the invention, attachment 920 and support strap 940 are manufactured as part of the inner layer of the base sleeve (e.g. 510 in FIG. 21A). Alternatively, attachment 920 may be sewn on to the base sleeve or attached by other attachment means as described above.

In an exemplary embodiment of the invention, support strap 940 comprises fasteners 930 at its ends (e.g. as described above regarding fasteners 520) to enable support strap 940 to be fastened around a limb

FIG. 21B is a schematic illustration of deployment of sleeve 900 with additional support according to an exemplary embodiment of the invention. In an exemplary embodiment of the invention, the base part of sleeve 900 is deployed around a user's limb, and support strap 940 is deployed around the user's limb at a higher position (i.e. closer to the user's trunk) than the base part, for example the base sleeve may be deployed on a user's leg below a user's knee with support strap 940 deployed above the user's knee. Alternatively, sleeve 900 may be deployed on other limbs of the user or in other positions.

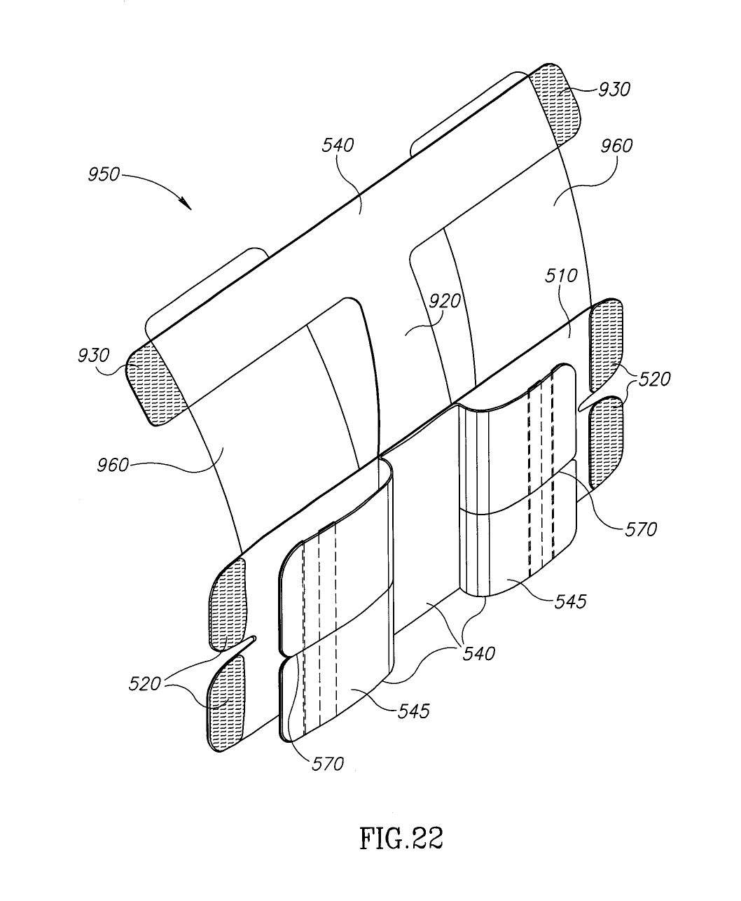

FIG. 22 is a schematic illustration of a sleeve 950 with protectors 960 for protecting a user's joint according to an exemplary embodiment of the invention. In an exemplary embodiment of the invention, sleeve 950 is manufactured with additional flaps which extend upward from a base sleeve, for example sleeve 500. Optionally, protectors 960 are manufactured as an integral part of sleeve 950. Alternatively, protectors 960 are sewn on to sleeve 950 or attached by other attachment means as described above. In some embodiments of the invention, protectors 960 are manufactured by strong material, for example leather, in order to protect the user's joint from harm. Optionally, when deploying sleeve 950, strap 940 encircles protectors 960 and positions them over the user's joint.

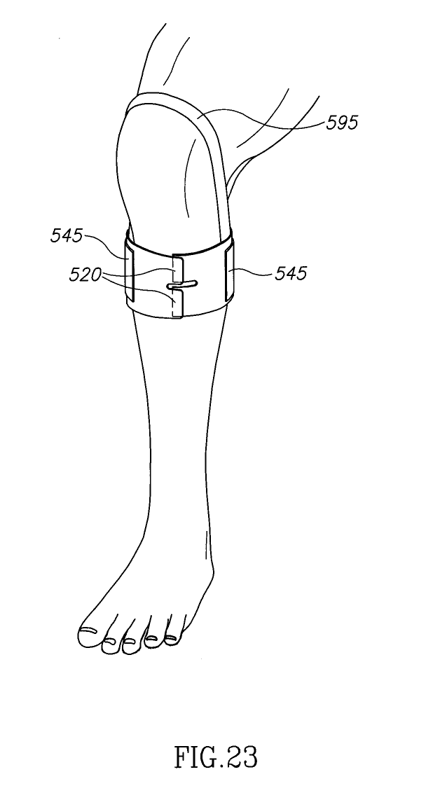

FIG. 23 is a schematic illustration of deployment of a sleeve with additional support according to an exemplary embodiment of the invention. In an exemplary embodiment of the invention, a user deploys a sleeve, for example sleeve 500, on the user's calf while the user is situated in a sitting position, for example during an airplane trip. Optionally, a strap 595 is deployed from one side of the user's calf to the other side, over the user's knee in order to add support to sleeve 500 and prevent it from sliding downward due to the weight of the circulation enhancement device. In some embodiments of the invention, strap 595 is attached using hook and loop fasteners or other detachable fastener means to both sides of sleeve 500, thus allowing maximum flexibility in adjusting strap 595. Alternatively, at least one side is connected with non-detachable means, for example sewn together, to prevent strap 595 from being misplaced. In some embodiments of the invention, both sides are non-detachable.

In some embodiments of the invention, strap 595 is elastic to provide for different size users. Alternatively, the position of sleeve 500 is adjusted or the position of attachment of strap 595 is adjusted, when deploying sleeve 500 with strap 595.

In some embodiments of the invention, a user may wear more than one circulation enhancement device at the same time, for example on each leg and/or on the user's arms. Optionally, each of the circulation enhancement devices may be of a different type, for example a lighter model may be worn on the arm. Additionally, different sleeves may be used according to the limb dealt with and or the activity performed by the user, for example one model of a sleeve may be used for walking and another may be used for sitting.

It should be appreciated that the above described methods may be varied in many ways, including omitting or adding steps, changing order of the steps and the type of devices used. It should be appreciated that different features may be combined in different ways. In particular, not all the features shown above in a particular embodiment are necessary in every embodiment of the invention. Further combinations of the above features are also considered to be within the scope of some embodiments of the invention. Section headings are provided for assistance in navigation and should not be considered as necessarily limiting the contents of the section.

It will be appreciated by persons skilled in the art that the present invention is not limited to what has been particularly shown and described hereinabove. Rather the scope of the present invention is defined only by the claims which follow.

1. A sleeve for use with a circulation enhancement device, the sleeve substantially encircles a user's leg at the region of the user's calf muscle and comprises two end-straps of non-stretchable material configured to connect to opposite lateral sides of said circulation enhancement device to enable intermittent tightening and relaxing of said sleeve around the user's leg by pulling and releasing both end straps, said two end straps extend in opposite directions along a common longitudinal axis;
wherein at least one of said two end straps is connectable to said circulation enhancement device by passing through a loop in one of said opposite lateral sides of said circulation enhancement device; and
wherein said at least one end strap comprises attachment means configured to be attached at various positions along said sleeve, after passing through the loop, to enable adjusting the sleeve to fit the user's leg.

2. A sleeve according to claim 1 wherein both said end straps are connectable to the device, each by passing through a respective loop in one of said opposite lateral sides of said circulation enhancement device and wherein each end strap is provided with attachment means configured to be attached at various positions along said sleeve.

3. A sleeve according to claim 1 wherein said at least one end strap further comprises attachment reception means compatible with said attachment means, said attachment means and attachment reception means are on the same surface of said at least one end strap.

4. A sleeve according to claim 3 wherein said attachment means and attachment reception means are hook and loop fasteners.

5. A sleeve according to claim 1 wherein the sleeve is an open sleeve adapted to be closed around the user's leg to form a closure around the user's leg.

6. A sleeve according to claim 5 wherein the sleeve comprises fastening means to enable closing the sleeve to form said closure.

7. A sleeve according to claim 6 wherein said fastening means are hook and loop fasteners.

8. A sleeve according to claim 1 further comprising an elastic extension attached to said straps to assist the user in deploying the sleeve around the user's leg.

9. A sleeve according to claim 1, wherein said sleeve comprises a middle section configured to interface with the user's calf muscle.

10. A sleeve according to claim 9 wherein said middle section has a piece cut out from its center.

11. A sleeve according to claim 9, wherein said middle section is flat.

12. A sleeve according to claim 9, wherein said middle section is shaped like a piece of a sphere.

13. A sleeve for use with a circulation enhancement device, the sleeve comprising:
a first layer that is adapted to substantially encircle a user's leg at the region of the user's calf;
a second layer that is adapted to interface said circulation enhancement device; and
a connection between said first layer and said second layer at a preset position;
wherein said second layer comprises two straps of non-elastic material configured to connect to opposite lateral sides of said circulation enhancement device, each by passing through a respective loop in one of said opposite lateral sides of said circulation enhancement device, to enable intermittent tightening and relaxing said sleeve around the user's leg by pulling and releasing both straps, said two straps extend in opposite directions along a common longitudinal axis; and
wherein said two straps comprise attachment means for attaching the straps to at least one of said first and second layers, after passing through the loop.

14. A sleeve according to claim 13 wherein the sleeve is an open sleeve adapted to be closed around the user's leg to form a closure around the user's leg.

15. A sleeve according to claim 13, wherein said first layer comprises edges with an elastic lining along the edges.

16. A sleeve according to claim 13, wherein said first layer and said second layer are connected by sewing.

17. A sleeve according to claim 13, wherein said first layer and said second layer are connected by snaps.

18. A sleeve according to claim 13, wherein said first layer and said second layer are connected by glue.

19. A sleeve according to claim 13 wherein said first layer and said second layer are connected by welding.

20. A sleeve according to claim 13, wherein said second layer is attached to various positions along at least one of the first and second layers or the second using hook and loop fasteners.

21. A sleeve according to claim 13, wherein said first layer comprises two opposite ends provided with fastening means to enable tightening the sleeve around the user's leg.

22. A sleeve according to claim 21, wherein said fastening means are hook and loop fasteners.
